(19) United States
(12) Patent Application Publication
(10) Pub. No.: US 2024/0299294 A1
(43) Pub. Date: Sep. 12, 2024

(54) TARGETED DELIVERY OF EFFECTIVE THERAPEUTIC MEDICATIONS TO THE RESPIRATORY TRACT

(71) Applicant: Amphastar Pharmaceuticals, Inc., Rancho Cucamonga, CA (US)

(72) Inventors: Jack Yongfeng ZHANG, Rancho Cucamonga, CA (US); Mary Zi-ping LUO, Rancho Cucamonga, CA (US); Ying WANG, Rancho Cucamonga, CA (US); Yi XIA, Rancho Cucamonga, CA (US); Jie Fei DING, Rancho Cucamonga, CA (US); Selina SU, Rancho Cucamongo, CA (US); Qingxia HAN, Rancho Cucamonga, CA (US)

(21) Appl. No.: 18/115,106

(22) Filed: Feb. 28, 2023

Related U.S. Application Data
(63) Continuation-in-part of application No. 17/910,405, filed on Sep. 9, 2022.
(60) Provisional application No. 63/019,997, filed on May 4, 2020.
(60) Provisional application No. 63/019,981, filed on May 4, 2020.
(60) Provisional application No. 63/019,978, filed on May 4, 2020.
(60) Provisional application No. 63/019,974, filed on May 4, 2020.
(60) Provisional application No. 63/013,405, filed on Apr. 21, 2020.

(51) Int. Cl. A61K 9/00; A61K 31/4706; A61K 47/10; A61P 31/14

(52) U.S. Cl. CPC A61K 9/008; A61K 31/4706; A61K 47/10; A61P 31/14

(57) ABSTRACT

A method for delivering a medication for treatment of a pulmonary disease depending on location(s) of infection lesion(s) within a respiratory tract of a patient is disclosed. The method includes administering metered doses of the medication to the patient having the pulmonary disease by a metered dose inhaler (MDI) actuator having a function of controlling drug particle delivery characteristics. The medication is effective for treating the pulmonary disease. A therapeutically effective amount of medication for treating the pulmonary disease is administered by various metered doses of the medication. The medication includes an active pharmaceutical ingredient (API) associated with treatment of a pulmonary disease. The medication includes a propellant, a co-solvent, and a surfactant. The API is dissolved in the propellant at a pre-determined ratio, with or without a co-solvent. The medication is administered via metered-dose inhalation.

### CROSS-REFERENCE TO RELATED APPLICATIONS AND INCORPORATION BY REFERENCE TO ANY PRIORITY APPLICATIONS

The present application claims the benefit of priority to and is a continuation-in-part of U.S. patent application Ser. No. 17/910,405, filed Sep. 9, 2022, which is U.S. National Stage Application under 35 U.S. § 371(c) of International Patent Application No. PCT/US2021/028490, filed Apr. 21, 2021, which claims priority to U.S. Provisional Application No. 63/013,405, filed Apr. 21, 2020, U.S. Provisional Application No. 63/019,974, filed on May 4, 2020, U.S. Provisional Application No. 63/019,978, filed on May 4, 2020, U.S. Provisional Application No. 63/019,997, filed on May 4, 2020, and U.S. Provisional Application No. 63/019,981, filed on May 4, 2020, the disclosures of which are all hereby incorporated by reference in their entireties.

### TECHNICAL FIELD

The present invention relates to methods for treatment of a pulmonary disease. In some examples, the pulmonary disease is a respiratory disease, e.g., COVID-19. The medication is delivered by administration from a specially designed metered dose inhaler (MDI), which has a function of controlling drug particle delivery characteristics. In this way, the medication is delivered at various specific locations within a respiratory tract, including toward infection lesions caused by the pulmonary disease in the deep lung, including alveoli. The medication includes effective anti-viral therapeutic agents, such as hydroxychloroquine (HCQ). Disclosed methods allow for a reduced systemic dose of HCQ, but provide a high local concentration of HCQ in the respiratory tract and cause relatively fewer adverse drug events and lower overdose toxicity risk, while providing increased therapeutic efficacy.

### BACKGROUND

SARS-CoV-2 and/or its variants (CoV2) have been identified as responsible for causing COVID-19. CoV2 can be airborne and enter the human body through inhalation, such as through the nose and/or mouth. Consequently, CoV2 can travel in the respiratory tract into various regions of the airways, including into the lungs and alveoli. Severe or prolonged infection can also cause severe alveolar damage, which is a major cause of morbidity and mortality in certain affected COVID-19 patients. Notably, the human host receptor angiotensin-converting enzyme 2 (ACE2) has been identified in academic literature as the main receptor for CoV2 to enter and infect cells through endosomal entry, where CoV2 uses its distinctive spike proteins to bind to ACE2 receptors.

Hydroxychloroquine (HCQ) is recognized in academia and industry as having multiple antiviral mechanisms. Of these mechanisms, HCQ's ability to effectively block viral entry has the most accumulated evidence, including evidence demonstrating therapeutic efficacy for treatment of COVID-19. For example, published studies have indicated that HCQ inhibits viral entry by directly binding with the virus receptor ACE2 and/or by reducing glycosylation of ACE2, which is important for virus spike protein binding. In addition, increasing endosome and/or lysosome pH values required for viral cell fusion and/or entry are additional possible antiviral mechanisms involving HCQ. Besides these described example direct antiviral mechanisms for COVID-19 treatment, HCQ is also an effective anti-inflammatory drug that has shown its capability to mitigate inflammation in the respiratory system caused by overactive immune system response.

Due to HCQ's potential to treat COVID-19, both hydroxychloroquine (HCQ) and chloroquine (CQ) oral tablets have been used as an “off-label” (e.g., not officially sanctioned) oral treatment for combating CoV2 and/or its variants. Regardless of the prevalence of such “off-label” usage, the clinical benefits of HCQ oral tablets in treating COVID-19 still has not been currently proven. In addition, such orally-administered tablets tend to present significant efficacy and safety limitations due to only providing a relatively low bioavailability. As a result, healthcare providers may consider increasing HCQ dosage levels; however, relatively high doses of HCQ can result in serious cardiovascular complications in certain patients, further complicating HCQ therapy overall. In some cases, oral clinical dose regimens include 800 milligrams (mg) of HCQ sulfate on the first day of treatment to be followed by 400 milligrams (mg) daily for four to seven days of total treatment based on clinical evaluation. Since HCQ has a relatively tight therapeutic index and can be extremely toxic in overdose, a series of cardiovascular toxicity-related events may occur in overdose situations. In some cases, such events may include (but are not limited to): QT prolongation, sodium-channel inhibition, ventricular arrhythmias, conduction blockade, as well as cardiovascular collapse.

Specifically, regarding the bioavailability provided by oral HCQ tablets, only about 0.07% of a HCQ oral tablet dose may be distributed to the plasma and ultimately reach the lungs and alveoli. This relatively low concentration of HCQ delivered to infected areas is typically ineffective in successfully treating or curing COVID-19. In addition, the described oral dose delivery methods distribute administered medication systemically, meaning throughout the body, resulting in the undesirable spreading of medication thinly and potentially to non-infected regions within the body as well. As a result, orally ingested HCQ medication typically cannot attain a therapeutically effective concentration in the respiratory system including the lungs and alveoli for successful treatment of COVID-19.

Regarding HCQ delivery to patients using methods other than oral administration, various techniques have been attempted earlier, none indicating success in terms of reaching difficult-to-reach regions of the respiratory tract, such as the deep lungs and/or the peripheral lungs. For example, U.S. Pat. No. 6,572,858 (the “'858 patent”) entitled “Uses for Anti-Malarial Therapeutic Agents” relates to methods for treating an inflammatory condition, such as asthma, in an animal including administering (such as locally to the area of inflammation) to the animal, an anti-inflammatory effective amount of an anti-malarial compound, such as HCQ. The '858 patent describes that “[f]or pulmonary delivery, a therapeutic composition of the invention is formulated and administered to the patient in solid or liquid particulate form by direct administration e.g., inhalation into the respiratory system.” Id., Col. 14, Lines 3-6. More particularly, the '858 patent uses a “traditional nebulizer which works in a mechanism similar to the familiar perfume atomizer” Id., Col. 14, Lines 54-56. However, the studies disclosed by '858 patent fail to adequately show production of HCQ particles with sufficiently small sizes that may travel into regions of the respiratory tract, such as the deep lungs and/or the peripheral lungs. Consequently, the need for such local or lung-targeted delivery methods for a respiratory disease, such as COVID-19, remains currently unmet.

Similarly, EP3892275 (the “'275 European Patent Application”) describes a “method of treating Severe Acute Respiratory Syndrome-Corona Virus (SARS-COV) in a patient in need thereof comprising administering to the patient's lungs a therapeutically effective amount of hydroxychloroquine (HCQ) or its metabolites by aerosolization” where “the SARS-COV [produces] COVID-19.” The '275 European Patent Application discloses that “[s]tudies conducted in vitro at a cellular level have shown that HCQ is a highly potent antiviral drug, including for COVID [ . . . etc.] If administered as an aerosol in patients affected by COVID related lung diseases, HCQ could reach the same pulmonary sites as HCQ circulating in the blood.” However, studies disclosed by the '275 European Patent Application may fail to adequately disclose complete nonclinical animal studies indicating dispersion of aerosolized particulate matter having specific desirable sizes capable of progressing into regions of the respiratory tract infected by CoV2.

As described above, clinical experience has shown that higher orally-administered doses (e.g., in tablet form) are likely to be excessively toxic. This is one of the reasons why the efficacy of HCQ oral tablet therapy remains controversial and, perhaps, may be one of the reasons for its unproven benefits for treatment of COVID-19. In addition, previous HCQ inhalation therapy attempts, such as those described by the '858 patent and '275 European Patent Application briefly discussed above, fail to show methods for sufficiently controlling drug particle delivery characteristics to deliver aerosolized HCQ medication to relatively hard-to-reach regions of the respiratory tract, such as the deep lungs and/or the peripheral lungs. Various aspects of the present disclosure recognize that currently available methods for oral and/or pulmonary administration of HCQ are still inadequate for effective treatment of COVID-19.

Accordingly, there is a current unmet need for methods to safely administer HCQ to a patient in a manner that reaches the respiratory tract including upper and lower airways as well as harder-to-reach regions of the lungs, such as the alveoli. Such targeted delivery of medication directly to the respiratory tract is expected to enable a relatively lower dose of HCQ, which may still be sufficient to provide therapeutic efficacy to treat the disease at the infected sites.

The present disclosure describes methods for targeted delivery of aerosolized HCQ formulations within the respiratory tract, defined as including upper and lower airways as well as relatively hard-to-reach regions such as the alveoli. More particularly, disclosed aspects relate to delivery of HCQ by metered-dose inhalation by a metered-dose inhaler (MDI). Disclosed methods of medication delivery enable delivery of HCQ medication to treat infection lesions throughout the respiratory tract, including the deep lungs and/or the peripheral lungs and/or the alveoli. Disclosed aspects relate to an inhalable lung-targeted medication, which allows the therapeutic dose to be significantly lower than that of the oral delivery to achieve the same efficacy. Notably, disclosed study results demonstrated that, when compared to standard oral dose regimes, the required effective dose using the disclosed respiratory tract delivery could be significantly smaller, such as at about a reduction of 100-150 times or more. Inhalation administration of HCQ by the disclosed aerosolized formulations thus reduce the possibility of potential adverse reactions caused by a relatively high oral dose of HCQ (total 2,400 to 3,600 mg in 5-7 days) and long half-life (123.5 days) for HCQ oral tablet delivery.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter described in this disclosure can be implemented as a method for delivering a medication for treatment of a pulmonary disease depending on location(s) of infection lesion(s) within a respiratory tract of a patient. The respiratory tract is defined as comprising an upper airway and a lower airway including alveoli in a relatively deep portion of lungs. The method includes administering a metered dose (such as including one or more metered doses) of the medication to the patient having the pulmonary disease by a metered dose inhaler (MDI) actuator having “a function of controlling drug particle delivery characteristics.” In addition, the medication is effective for treating the pulmonary disease. A therapeutically effective amount of medication for treating the pulmonary disease is administered by the metered dose. In some examples, the pulmonary disease is COVID-19 and the virus is SARS-CoV-2 and/or its variants.

The MDI actuator facilitates self-administration of the medication by the patient. When a patient actuates the MDI by compressing a canister toward a metering chamber to release a metered dose of the medication from the canister into the expansion chamber, the metered dose emerges through an MDI actuator as an aerosolized spray that is then inhaled by the patient. In this way, the MDI actuator has a “function of controlling drug particle delivery characteristics” for delivering a metered dose of the medication to the patient. The drug particle delivery characteristics of an MDI typically includes valve delivery (referring to an amount of formulation released per actuation), delivered dose uniformity, particle size distribution, and/or spray pattern. More specifically, the “function of controlling drug particle delivery characteristics” is defined by the orifice of the MDI actuator. More particularly, the orifice has a specific size that aerosolizes a formulated medication with specific particle size distribution and/or delivery characteristics to exit the MDI actuator at a desired speed or velocity. Consequently, by actuation of the MDI actuator, the medication is aerosolized and delivered at a specific amount onto a specific portion of the respiratory tract and lungs for a desired use of the medication.

Specifically, the actuator orifice is sized to control the drug particle delivery characteristics of fine and extra fine particles included in a dose. In this way, certain defined quantities of drug particles are delivered within the respiratory tract of the patient based on their respective size to treat infection lesions caused by the pulmonary disease. In some examples, the medication is formulated to include an active pharmaceutical ingredient (API), a propellant, a co-solvent, and a surfactant. The API is dissolved in the propellant at a pre-determined ratio, with or without a co-solvent. The medication is administered to the patient via metered-dose inhalation. In some examples, the API in the medication may be hydroxychloroquine (HCQ), and the metered dose of the medication is provided at various specific dosage ranges, including from about 0.05 mg to about 1.00 mg, about 0.1 mg to about 0.5 mg, or about 0.1 mg to about 0.4 mg.

Another innovative aspect of the subject matter described in this disclosure can be implemented as a method of treating a pulmonary disease caused by a virus in a respiratory tract of a patient depending on location(s) of infection lesion(s) caused by the pulmonary disease. In some examples, the pulmonary disease is COVID-19 and the virus is SARS-CoV-2 and/or its variants. The respiratory tract is defined as comprising an upper airway and a lower airway including alveoli in a relatively deep portion of lungs of the patient. The method includes administering, by a metered dose inhaler (MDI) actuator having a function of controlling drug particle delivery characteristics, an anti-viral medication to the patient. The anti-viral medication includes hydroxychloroquine (HCQ) in a free base thereof or a pharmaceutically acceptable salt thereof. A therapeutically effective amount of the anti-viral medication for treating the pulmonary disease is administered by one or more metered doses of the anti-viral medication. In some embodiments, the MDI actuator has an orifice having a diameter of 0.18 mm to 0.25 mm, which associated with HCQ drug particle delivery.

In this way, the MDI actuator (more specifically, the orifice of the MDI actuator) has a function of: (1) controlling drug particle delivery characteristics for delivery of at least 70% of a delivered dose of the HCQ medication, which includes fine particles of the medication that reach, as measured in a cascade impactor, to “Stage 3 to Stage 7 and beyond;” and, (2) targeting the respiratory tract of the patient, from the upper airway to the lower airway including alveoli located, for example, in the deep and the peripheral lungs. “Stage 3 to Stage 7 and beyond” is based on a cascade impactor particle size distribution (PSD) of a respiratory tract. The fine particles of the medication have particle diameter sizes of less than 4.7 μm.

In addition, the MDI actuator has a function of controlling drug particle delivery characteristics for delivery of at least 30% of a delivered dose of the HCQ medication, which includes extra fine particles of the medication that reach, as measured in a cascade impactor, “Stage 6 to Stage 7 and beyond,” including targeting relatively deep regions of lungs of the patient including where alveoli are located in the peripheral lungs. “Stage 6 to Stage 7 and beyond” is based on a cascade impactor PSD of a respiratory tract. The extra fine particles of the medication have particle diameter sizes of less than 1.1 μm. A single metered dose of the anti-viral medication from the MDI is capable of delivering aerosolized particles of the anti-viral medication having particle diameters of less than 1.1 μm.

The method of delivery of the antiviral medication into the relatively deep portion of lungs of the patient allows for a reduced systemic dose of the medication, provides a relatively high local concentration of the medication in the respiratory tract, and thereby causes fewer adverse drug events and lowers overdose toxicity risk, while providing increased therapeutic efficacy. The medication is formulated with HCQ, a propellant, a co-solvent, and a surfactant. API is dissolved in the propellant at a pre-determined ratio, with or without a co-solvent.

The API includes HCQ free base or pharmaceutically acceptable salt which can be converted into HCQ free base, which is formed from conversion of HCQ sulfate, and is provided at various concentration levels, such as at about 0.25% to 1.50% (w/w), about 0.40% to 0.70% (w/w), and about 0.60% to 0.70% (w/w). The co-solvent includes ethanol, and is provided at various concentration levels, such as at about 1% to about 15% (w/w), about 4.00% to 8.00% (w/w), or about 5.50% to 6.50% (w/w). The surfactant includes poly(ethylene glycol)-block-poly(propylene glycol)-block-poly(ethylene glycol) (Poloxamer 124) provided at various concentration levels including at about 0% to 1% (w/w), about 0.01% to about 0.2% (w/w), and about 0.01 to about 0.05% (w/w). The propellant includes 1,1,1,2-Tetrafluoroethane (HFA 134a) provided at various concentration levels including at about 80% to about 97% (w/w), about 93.00% to about 96.00% (w/w), and about 93.00% to about 94.00% (w/w), where “w/w” denotes weight by weight, and is based on a total weight of the medication, which is formulated as a true solution. In addition, the medication has a favorable half maximal effective concentration (EC₅₀), including that of hydroxychloroquine (HCQ), in a free base thereof, or a pharmaceutically acceptable salt thereof. Half maximal effective concentration (EC₅₀) is the concentration or dose effective in producing 50% of the maximal response. Specifically, here, “favorable half maximal effective concentration (EC₅₀)” is defined in some examples shown in Table 15, which indicates that the estimated alveolar lining fluid (ALF) HCQ concentration was highest at the first sampling point (10 minutes) at 476 μM, and remained as high as 188 μM after 6 hours post dosing, which still greatly exceeds the EC₅₀ of HCQ for viral inhibition (0.72-17.31 μM). Exceeding the EC₅₀ of HCQ for viral inhibition of CoV2 is defined by the present disclosure as being “favorable.”

The medication was applied to (such as by being tested in) a non-clinical animal model including mice. More specifically, the non-clinical animal model included the MDI actuator inserted into a rodent (mouse) inhaler tank, where multiple mice were placed in mouse restraints with their noses exposed to a main section of the rodent inhaler tank (breathing tank) loaded with the medication. Disclosed methods include administering an amount of HCQ according to a human equivalent dose. Delivered particles of HCQ reach the respiratory tract and lungs of treated mice in the non-clinical animal model and demonstrated “well-tolerated” toxicity and safety profiles. More specifically, “well-tolerated” toxicity is defined as shown in Example 12 as having a no-observed-adverse-effect-level (NOAEL) dose that is 6-fold and 15-fold higher than the intended human dose. In addition, within a first measurement point taken between 5- and 10-minutes post-dosing via the breathing tank of the non-clinical animal model, at least 5% of the pre-determined amount of HCQ enters into lungs of treated mice in the non-clinical animal model.

Medication delivered into lungs of treated mice in the non-clinical animal model reaches and maintains an estimated alveolar (ALF) HCQ concentration above the HCQ antiviral EC₅₀ concentration against SARS-CoV-2 for no less than 6 hours. In addition, the medication provided to treated mice in the non-clinical animal model has a plasma elimination half-life of not less than 4 hours and has a concentration level in mouse plasma that is at least 25 times lower than that observed in lungs of treated mice in the non-clinical animal model. Concentration levels of the medication in mouse plasma demonstrate no accumulated effect after 5-day repeated doses as indicated by maximum concentration (Cₘₐₓ) and area under the curve (AUC).

Details of various implementations of the subject matter described in this disclosure are set forth in the accompanying drawings and the description below. Other features, aspects, and advantages will become apparent from the description, the drawings, and the claims. Note that the relative dimensions of the following figures may not be drawn to scale.

### BRIEF DESCRIPTION OF THE DRAWINGS

The foregoing summary, as well as the following detailed description of the invention, will be better understood when read in conjunction with the appended figures. For the purpose of illustrating the invention, the figures demonstrate embodiments of the present invention. It should be understood, however, that the invention is not limited to the precise arrangements, examples, and instrumentalities shown. A full and enabling disclosure of the present disclosure, including the best mode thereof, directed to one of ordinary skill in the art, is set forth more particularly in the remainder of the specification, which makes reference to the appended drawings in which:

FIG. 1 depicts a cross-sectional view of a metered dose inhaler (MDI) actuator;

FIG. 2 depicts a graph showing particle size distribution (PSD) over an orifice diameter of the MDI actuator shown in FIG. 1;

FIGS. 3A to 3D depict bar charts showing efficacy of various configurations of the MDI actuator of FIG. 1;

FIG. 4 depicts a rodent inhaler breathing tank prepared for testing medications delivered by the MDI actuator of FIG. 1;

FIG. 5 depicts a graph showing the pharmacokinetic (PK) profile of HCQ in mouse lungs post inhalation treatment;

FIGS. 6A to 6D depict multiple graphs showing lung HCQ concentrations in mice after treatment;

FIG. 7 depicts a table showing Cₘₐₓ, tₘₐₓ, and AUCs of mice lung HCQ exposure after Low, Mid, and High Doses of HCQ Delivery;

FIG. 8 depicts a graph showing Mouse Plasma HCQ Pharmacokinetic at Single, High Dose;

FIG. 9 depicts a table for a toxicokinetic (TK) Summary of the 5-Day Repeated Dose;

FIG. 10 is a table showing alveolar lining fluid (ALF) determination of hydroxychloroquine (HCQ) deposited in mouse lungs; and

Repeat use of references characters in the present specification and drawing is intended to represent same or analogous features or elements of the disclosure.

### DETAILED DESCRIPTION

It is to be understood by one of ordinary skill in the art that the present discussion is a description of example embodiments only and is not intended as limiting the broader aspects of the present disclosure.

The present disclosure is directed to a method for delivering a medication for treatment of a pulmonary disease depending on location(s) of infection lesion(s) within a respiratory tract of a patient. The respiratory tract of the patient is defined as comprising an upper airway and a lower airway including alveoli in a relatively deep portion of lungs of the patient. The method includes administering a metered dose of the medication to the patient having the pulmonary disease by a metered dose inhaler (MDI) actuator having a function of controlling drug particle delivery characteristics, where “a function of controlling drug particle delivery characteristics” is defined by the MDI actuator, which has an orifice with a specific size that aerosolizes a formulated medication with specific particle size distribution and/or delivery characteristics to exit the MDI actuator at a desired speed or velocity. Consequently, by actuation of the MDI actuator, the medication is aerosolized and delivered at a specific amount onto a specific portion of respiratory tract and lungs for a desired use of the medication. The medication is effective for treating the pulmonary disease. In addition, a therapeutically effective amount of the medication for treating the pulmonary disease is administered by a metered dose of the medication.

The word “exemplary” is used herein to mean “serving as an example, instance, or illustration.” Any embodiment described herein as “exemplary” is not necessarily to be construed as preferred or advantageous over other embodiments. Likewise, the term “embodiments” does not require that all embodiments include the discussed feature, advantage, or mode of operation.

Unless otherwise defined herein, scientific, and technical terms used in connection with embodiments of present disclosure shall have the meanings that are commonly understood by those of ordinary skill in the art. Nomenclatures used in connection with, and techniques described herein are those known and commonly used in the art. Also, descriptions of well-known functions and constructions are omitted for clarity and conciseness.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the terms “comprising” as used herein are synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the disclosure, but instead as merely intended to highlight alternative or additional features that may or may not be utilized in a particular embodiment of the disclosure. In addition, the term “comprising” is to be interpreted synonymously with the phrases “having at least” or “including at least.” When used in the context of a process, the term “comprising” means that the process includes at least the recited steps but may include additional steps. When used in the context of a compound, composition or device, the term “comprising” means that the compound, composition, or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Additionally, the phrase “consisting essentially of” will be understood to include those elements specifically recited and those additional elements that do not materially affect the basic and novel characteristics of the claimed technology. The phrase “consisting of” excludes any element not specified.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

“Treat” or “treating” or “treatment” refers to any type of action that imparts a modulating effect, which, for example, can be a beneficial effect, to a subject afflicted with a disorder, disease or illness, including preventing the manifestation of disease states associated with the condition, improvement in the condition of the subject (e.g., in various symptoms or in the disease), delay or reduction in the progression of the condition, and/or change in clinical parameters, disease or illness, curing the illness, etc.

The “patient” or “subject” treated as disclosed herein is, in some embodiments, a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” as used herein includes, but is not limited to, humans, non-human primates, cattle, sheep, goats, pigs, horses, cats, dog, rabbits, rodents (e.g., rats or mice), monkeys, etc. Human subjects include neonates, infants, juveniles, adults, and geriatric subjects. Human subjects may have an age in years ranging from between 0 and 6, 6 and 12, 12 and 18, or over 18 (e.g., adults).

As used herein, the term “percent weight by weight” (alternatively: “% w/w”, % (w/w), “weight by weight percent”, or other similar language) when referring to an impurity, is the weight of the impurity divided by the weight of an API present, multiplied by 100%. For example, the percent weight by weight (or % w/w) of impurity A when 5 grams of impurity A resides in a composition having 100 grams of API B is 5% (e.g., 5 g A/(100 g API B)×100%).

As used herein, the term “weight percent” (or “wt. %” or similar language) when referring to a component, is the weight of the component divided by the weight of the composition that includes the component, multiplied by 100%. For example, the weight percent of component C when 5 grams of component C is present in 95 grams of component D is 5% (e.g., 5 g C/(5 g C+95 g D)×100%).

As used herein, the term “mole percent” (or “mole %” or similar language) when referring to a component, is the number of moles of the component divided by the total number of moles of the composition that includes the component, multiplied by 100%. For example, the mole percent of component C when 5 moles of component C are present in 95 moles of component D is 5% (e.g., 5 moles C/(5 moles C+95 moles D)×100%).

As used herein, when the term “collectively or individually” (and variations thereof) modifies an amount of a component or components (e.g., a concentration) of multiple component composition, this usage means that each individual component may be provided in the amount disclosed or that combined amount of components may be provided in the amount disclosed. For example, if agents A and B are referred to as, collectively or individually, being present in a composition at a concentration of 5 mg/mL, that means that A may be at 5 mg/mL in the composition (individually), B may be at 5 mg/mL in the composition (individually), or the combination of A and B may be present at a total of 5 mg/mL (A+B=5 mg/mL, e.g., collectively). Where A is present at 5 mg/mL, B may be absent. Where B is present at 5 mg/mL, A may be absent. Alternatively, where both A and B are present, A may be at 5 mg/mL (individually) and B may be at 5 mg/mL (individually), totaling 10 mg/mL (collectively).

When referring to a variable, the terms “or ranges including and/or spanning the aforementioned values” (and variations thereof) is meant to include any range that includes or spans the aforementioned values. For example, for the concentration of an ingredient, when the concentration of the ingredient is expressed as “1 g/ml, 5 g/ml, 10 g/ml, 20 g/ml, or ranges including and/or spanning the aforementioned values,” this includes each of the particular concentrations explicitly provided or concentration ranges for the ingredient spanning any of the particular concentrations, such as, from 1 g/ml to 20 g/ml, 1 g/ml to 10 g/ml, 1 g/ml to 5 g/ml, 5 g/ml to 20 g/ml, 5 g/ml to 10 g/ml, and 10 g/ml to 20 g/ml.

As used herein, the terms “about” or “approximately” are terms of degree that avoid strict numerical boundaries. In some instances, “about” or “approximately” may be taken to mean plus or minus 10% of the numerical value those terms modify. For example, about 5% may, in some circumstances, refer to a range of values between 4.5% and 5.4%.

“Pharmaceutically acceptable” or “physiologically acceptable” refer to compounds, salts, compositions, dosage forms and other materials which are useful in preparing a pharmaceutical composition that is suitable for veterinary or human pharmaceutical use.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutical composition,” “pharmaceutical formulation, or “formulation” refers to a formulation of a compound of the disclosure and a medium generally accepted in the art for the delivery of the biologically active compound to mammals, for example, humans. Such a medium includes all pharmaceutically acceptable excipients therefor.

“Effective amount” or “therapeutically effective amount” refers to an amount of a compound of the disclosure, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound of the disclosure which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the disclosure, and the age, body weight, general health, sex and diet of the patient. Such a therapeutically effective amount can be determined routinely by one of ordinary skill in the art having regard to their own knowledge, the state of the art, and this disclosure.

“Treatment” or “treating” or “treat” refers to an approach for obtaining beneficial or desired results including clinical results. Beneficial or desired clinical results may include the following: a) inhibiting the disease or condition (e.g., decreasing various symptoms resulting from the disease or condition, and/or diminishing the extent of the disease or condition); b) slowing or arresting the development of various clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival. Thus, in the context of the disclosed methods of treatment of COVID-19, the disclosed methods are intended to: (i) reduce severity, (ii) eliminate, or (iii) reduce severity and eliminate the pulmonary disease symptoms, such as COVID-19 symptom(s).

“Prevention” or “preventing” means any treatment of a disease or condition that causes the clinical symptoms of the disease or condition not to develop. Compositions may, in some embodiments, be administered to a subject (including a human) who is at risk or has a family history of the disease or condition.

“Hydroxychloroquine” (HCQ) means HCQ or a pharmaceutically acceptable salt thereof. An example is hydroxychloroquine sulfate, commercially available as Plaquenil®. Hydroxychloroquine has the chemical structure:

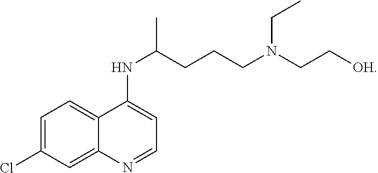 [SMILES: CCN(CCO)CCCC(C)Nc1ccnc2cc(Cl)ccc12]

Current commercially-available therapeutic treatment method may cause moderate to severe adverse drug effects (ADEs) while combating COVID-19, thereby resulting in an urgent and unmet medical need to develop drug products and formulations for treatment of COVID-19 caused by CoV2 and/or its variants. The presently disclosed embodiments uniquely address this unmet medical need by introducing a method of respiratory tract-targeted, non-systemic metered-dose inhaler (MDI) mediated delivery of an effective amount of the medication to treat a pulmonary disease, such as COVID-19. In this way, the medication can take effect directly on the infection lesion(s) throughout the respiratory tract, including upper and lower airways and alveoli in peripheral lungs. Corresponding pharmaceutical formulations and MDI actuators for self-administration are further disclosed herein.

### Introduction

### Particle Size Measuring Instrument and Aerodynamics

The cascade impactor has been used for accurately and precisely measuring the aerodynamic size distribution of inhaled drug products, such as those disclosed herein. Data provided by the cascade impactor indicates a direct link between the delivered mass and particle size of a pharmaceutical medication on different plates/stages as demonstrated in Table 1.

TABLE 1
Particle Sizes at Different Stages of the Respiratory Tract
Locaation as Measured by a Cascade Impactor

| | Typical Particle | Respiratory |
|---|---|---|
| Stage # | Size Range | Tract Location |
| 0 | 9.0 μm-10.0 μm | Oro-Nasopharynx |
| 1 | 5.8 μm-9.0 μm | |
| 2 | 4.7 μm-5.8 μm | Pharynx |
| 3 | 3.3 μm-4.7 μm | Trachea & Primary Bronchi |
| 4 | 2.1 μm-3.3 μm | Secondary Bronchi |
| 5 | 1.1 μm-2.1 μm | Terminal Bronchi |
| 6 | 0.65 μm-1.1 μm | Alveoli |
| 7 | 0.43 μm-0.65 μm | |

A metered-dose inhaler (MDI) can fulfill the goal of delivering a pharmaceutical formulation to the desired portions of respiratory tract and lungs. The MDI is a device that delivers a specific amount of medication to the lungs, in the form of a short burst of aerosolized medicine that can be self-administered by the patient via actuation of the MDI and subsequent inhalation of aerosolized medication dispersed from the MDI. Therefore, disclosed formulations, such as those including HCQ free base dispersed from an MDI, facilitates targeted delivery within a respiratory tract of a patient, including lung-targeted therapy for the treatment of the COVID-19 disease.

### Control of Particle Size Distribution

Disclosed medications (such as anti-viral medications) are dispersed from an MDI upon actuation and are aerosolized upon exit from the actuator within the expansion chamber as a high-velocity spray. The MDI actuator has a specially designed orifice diameter that has a function of controlling particle size distribution of the aerosolized medication. In some examples, a metered dose inhaler (MDI) was used. The MDI atomizes the disclosed formulations into fine particles. More particularly, the particle size distribution of the aerosolized formulation exiting the MDI is based on the orifice diameter of the MDI actuator. In this way, fine particles that have particle diameters of less than 4.7 μm can be measured in a cascade impactor and reach Stage 3 to Stage 7 and beyond, which can correspond with targeting the upper airway and the lower airway, including alveoli. In addition, particles with a diameter less than 1.1 μm can reach alveoli located in peripheral lungs, which correspond with measurement in cascade impactor Stages 6 to 7 and beyond.

Different technologies can be used to control particle size distribution (PSD). Many factors determine what proportion of an aerosolized dose is respirable, which is conventionally considered to be the drug mass composed of aerodynamic particles having diameters ≤5 μm. In conventional applications, undesirable loss of a 50% or more medicine of the metered dose to the actuator's mouthpiece and patient's oropharageal region is not uncommon. Regarding MDI operation, upon actuation of the MDI, the MDI's metering chamber becomes closed to the formulation reservoir and opens to the atmosphere, resulting in the expansion of the propellant-based formulation and atomization through the actuator orifice.

This invention uses a specially designed MDI actuator to control the HCQ delivery characteristics, where the respirable drug mass can be increased by decreasing the orifice diameter and/or the orifice cross-sectional area of the MDI actuator. The importance of the actuator orifice in determining drug delivery characteristics necessitates the tailoring of orifice dimensions to meet the requirements of each disclosed pharmaceutical medication formulation.

### Chemistry, Manufacturing and Controls (CMC) for Desired Aerosolized Formulation

To appropriately manufacture a pharmaceutical product, product characteristics, product testing, and specific manufacturing process, must be defined in order to ensure that the product is safe, effective and consistent between batches. These activities are known as CMC, chemistry, manufacturing, and control. All stages of the drug development life cycle, after drug discovery involve CMC.

The described embodiments disclose viable formulations for delivering anti-viral medications including HCQ locally to the respiratory tract and deep lungs effectively. Initially, when considering whether the disclosed medications should be formulated as a suspension or a true solution, studies were conducted that indicate that based on the testing results, especially the solubility characteristics of HCQ, that a true solution with HCQ free base is preferred over a suspension. A “true solution” is defined as a homogeneous mixture of two or more substances. In contrast, a suspension is a heterogeneous mixture of two or more substances where the solute particles do not dissolve and remain suspended throughout the mixture.

Disclosed embodiments relate to an MDI that delivers the metered amount of a medication, in the form of aerosolized drug particles, into a patient's mouth for subsequent inhalation into the respiratory tract and lungs of the patient. The medication is contained in a pressurized canister, such as an aluminum canister of the MDI. Propellant included in formulations with the medication drive the formulated drug from the canister and dispense, per actuation, as an aerosolized spray suitable for inhalation. Disclosed MDI devices use an aerosol formulation including an active pharmaceutical ingredient (API) and/or active moieties, and excipients, such as propellant(s), alcohol co-solvent(s), and surfactant(s). Disclosed propellants are selected from environmentally friendly propellants such as, 1,1,1,2-Tetrafluoroethane (HFA 134a) and/or heptafluoropropane (HFA 227). The disclosed API includes hydroxychloroquine (HCQ) formulated with HCQ free base or HCQ salt, which can be converted to HCQ base. Currently commercially available HCQ is mostly in the form of HCQ salt. In order to create a viable anti-viral formulation, presently disclosed methods relate to transforming HCQ salt to HCQ base that has a preferable solubility characteristics. The co-solvent used in the said MDI formulations is an alcohol, in particular ethanol. Typically, to create a MDI medication in solution, a co-solvent is added to the formulation to increase solubility of the drug or excipients. The surfactant used in the said MDI formulations is poly(ethylene glycol)-block-poly(propylene glycol)-block-poly(ethylene glycol) (Poloxamer 124). Typically, the addition of surfactants to an MDI formulation helps to prevent undesirable and/or excessive agglomeration of micronized particles and, together with co-solvent, contributes to control of particle size distribution of the formulation. In addition, the formulation is stabilized within the MDI to deliver medication particles to the respiratory tract with a significant portion of fine particles to reach the deep peripheral lungs or alveoli to combat respiratory pulmonary diseases, such as COVID-19.

### Effective Dose and Effective Delivery for Desired Dose

EC₅₀ is defined as the concentration or dose effective in producing 50% of the maximal response. The HCQ EC₅₀ for inhibition of CoV2 is 0.72-17.31 μM. However, during first two treatment days with conventional HCQ treatment via oral tablet administration, observed HCQ concentrations (Day-1 0.23 and 0.45 μM after the 1st and 2nd 400 mg dose in Day-1, respectively, and Day-2 0.67 μM) are below the EC₅₀ values for inhibition of CoV2. These results indicate why the low HCQ concentration in alveolar fluid caused by ingestion of HCQ oral tablets may be therapeutically insufficient and therefore likely suboptimal for antiviral treatment against CoV2. The presently disclosed embodiments relate to a method to deliver drug product via an MDI to generate particles in various sizes that can reach various portions of the respiratory tract comprising the upper airway and the lower airway including alveoli, with different delivery rates.

### Nonclinical Dose-Ranging, Toxicity, Pharmacokinetic, and Tolerability Study

A primary purpose of non-clinical studies is to discover target organ toxicity, pharmacokinetic (PK), dose-range, and tolerability after medication delivery, and to use this information for monitoring possible adverse event and/or toxicities in humans. During preclinical drug development, the proper analytical methods were validated to monitor the product.

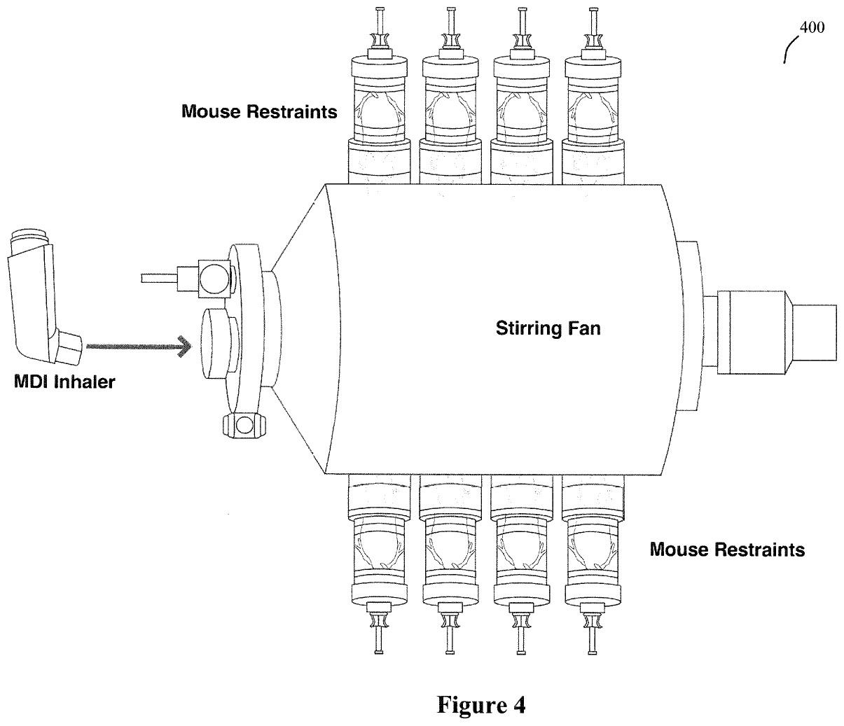

A nonclinical experiment including mice in mouse restraints connected to a breathing tank was used to determine target organ toxicity, pharmacokinetic, dose-range, and tolerability after the medication delivery. Mice cannot proactively inhale medications, but can breathe in aerosol of drug products, such as the presently disclosed anti-viral medications. A breathing tank was used for mice to breathe the aerosol of the intended drug product such as HCQ. The drug product was sprayed into a breathing tank 400 as shown in FIG. 4. The breathing tank is specially designed to facilitate delivery of the intended drug product to the mice and is made from stainless steel.

The results described further herein show that: (1) the HCQ particles can reach the respiratory tract and lungs of mice; and (2) the delivery can ensure that appreciable amount of medication is delivered to the intended target location and can last for hours and thereby achieve its intended therapeutic effect.

Both nonclinical toxicity and local tolerability should be appropriate for the intended usage for treating a pulmonary disease such as COVID-19 as caused by CoV2 and/or its variants. The nonclinical safety evaluation generally include a characterization of toxic effects with respect to target organs, dose dependence, relationship to exposure, and, when appropriate, potential reversibility. Further, tolerance should be determined at those sites that come into immediate contact with the medication as a result of the administration. Also, the site of administration of mice can be the same organ or tissue which is intended to be the therapeutic target of human. For example, the lung and respiratory tract is the therapeutic target and is also the site that comes into immediate contact with the administered anti-viral medication.

### Overview

Disclosed are methods for delivering a medication for treatment of a pulmonary disease depending on the location(s) of the infection lesion(s) within a respiratory tract of a patient. The respiratory tract is defined as comprising the upper airway and the lower airway including alveoli in a deep portion of the patient's lungs. The medication is administered as an aerosolized formulation from an MDI to treat a pulmonary disease, such as COVID-19. In this way, the medication can take effect right on the infection lesion(s) throughout the respiratory tract, including alveoli in peripheral lungs.

The MDI delivers a metered amount of the medication into a patient's mouth. As a result, the medication is then inhaled in the form of aerosolized drug particles into the respiratory tract and lungs of patient depending on intended target location(s). The medication is contained in a pressurized canister, such as an aluminum canister. Propellant included in the MDI drives the formulated drug from the canister to dispense, per actuation, an aerosolized spray suitable for inhalation. The MDI is specially designed to have a function of controlling drug particle delivery characteristics of the delivered medication. In particular, studies show that the actuator orifice diameter selected for use significantly determines the drug particle delivery characteristics of the atomized spray, as well as the fine particle fraction delivered. The disclosed embodiments investigated various actuators orifice sizes ranging between about 0.20 mm to about 0.42 mm.

In some embodiments, the medication can be self-administered using a handheld MDI actuator, which is specially designed such that it has a function of controlling drug particle delivery characteristics to deliver metered doses of a medication to a patient having a pulmonary disease. More specifically, via actuation of the MDI actuator, an aerosolized medication is delivered with a specific amount, onto a specific portion of respiratory tract and lungs to fit the intended use for a specific medication, such as treatment of COVID-19 caused by CoV2 with HCQ. In some embodiments, the MDI actuator has an orifice diameter of about 0.15 mm to 0.42 mm. In other embodiments, the MDI actuator has an orifice diameter of about 0.20 mm to 0.22 mm.

In some embodiments, one or more disclosed methods of administering the medication using the handheld MDI actuator indicates that: (i) about 33% to about 50% of particles measured as residing in Stages 6 and 7 of a cascade impactor are capable of reaching the alveoli to effectively combat CoV2 in the alveoli; and, (ii) about 49% to about 51% of particles measured as residing in Stages 3 to 5 of a cascade impactor can deliver HCQ from the trachea to the terminal bronchi in the upper and lower respiratory and thereby effectively treat CoV2 that may be located there. In some embodiments, the medication includes HCQ free base or its pharmaceutically acceptable salt thereof.

Thus, in some embodiments, the medication further comprises a propellant, which is HFA 134a. The HCQ is dissolved in the HFA 134a at a pre-determined ratio, with or without a co-solvent. In some embodiments, the said medication further comprises about 0.25% to about 1.50% (w/w) of HCQ; a co-solvent, such as an alcohol, of about 1.00% to 15.00% (w/w); a surfactant of about 0% to about 1%; a propellant of about 80.00% to about 97.00% (w/w); and wherein “w/w” denotes weight by weight. In some embodiments, the said medication comprises about 0.40% to about 0.70% (w/w) of HCQ; a co-solvent, such as an alcohol, of about 4.00% to about 8.00% (w/w); a surfactant of about 0.01% to about 0.2%; a propellant of about 93.00% to about 96.00% (w/w); and “w/w” denotes weight by weight.

In further embodiments, the medication comprises about 0.60% to about 0.70% (w/w) of HCQ, a co-solvent, such as an alcohol, of about 5.50% to about 6.50% (w/w), a surfactant of about 0.01% to about 0.05%; a propellant of about 93.00% to about 94.00% (w/w), where “w/w” denotes weight by weight. In some embodiments, in a single metered dose, at least about 30% of the medication has a particle diameter of less than about 1.1 μm, and the at least about 30% of the medication is capable of being delivered to the deep peripheral lungs or alveoli. Further, at least 70% of the said delivered dose are fine particles that have particle diameters of less than 4.7 μm reaching Stage 3 to 7 and beyond targeting the respiratory tract comprising the upper airway and the lower airway including alveoli in the peripheral lungs.

In some embodiments, the metered dose(s) of the formulated medication is delivered throughout the respiratory tract to treat the pulmonary disease directly on the infection lesion. In some embodiments, the targeted delivery of the medication reaches the deep peripheral lungs or alveoli and to a portion of the alveolar lining fluid, resulting in an efficacious, high local concentration for treating the pulmonary disease. In some embodiments, the therapeutically effective dose of the medication, such as that of HCQ, is administered by a metered dose. The medication formulated provides a dose of API per actuation (or per spray) of about 0.01 mg to about 1 mg of HCQ. In some embodiments, a single metered dose of the API, such as that of HCQ, is about 0.05 mg to about 1.00 mg, and about 0.20 mg to about 0.4 mg.

In some embodiments, the medication formulated provides a dose of API per actuation (or per spray) of about 0.2 to about 0.4 mg of HCQ. In some embodiments, a single metered dose of the API, such as that of HCQ, is about 0.35 mg to about 0.4 mg, or exactly 0.4 mg. In some embodiments, the medication was applied to a non-clinical animal model, such as that of mice, by administering certain amount of HCQ, according to the human equivalent dose, wherein the delivered particles can reach the respiratory tract, lungs, including deep peripheral lungs and demonstrated well-tolerated toxicity and safety profiles.

In some embodiments, the strength of HCQ administered is 0.2 mg/ml-0.4 mg/ml at a dose of 0.94 mcg-3.61 mcg, which reached the lungs of mice. This led a reduced systemic dose of HCQ at 4.9 ng/ml-162.9 ng/ml, while provided high local concentration of HCQ in mouse lungs 277.1 ng/g-9,527 ng/g (“ng/g” in lung is equivalent to “ng/ml” in plasma) during 5 min.-24 hours post dosing. In some embodiments, the concentration in the mouse lungs reached 3,365 ng/g-9,527 ng/g within at least 5 min. to 10 min. post dose, at the first sampling point.

In some embodiments, the delivery of the medication, such as HCQ, was determined as the effective concentration in alveoli lining fluid (ALF). The delivery rate was determined to be not less than 5.0% to 7.1% within at least 5 min. to 10 min. post dose, at the first sampling point. In some embodiments, the delivered medication in the mouse lungs can be detected for no less than 24 h. In some embodiments, the delivered medication has a half-life of 4.6 h-7.8 h.

In further embodiments, the medication can be used to treat disease having different levels of severity by varying the dosing regimen. For example, the number of doses of medication prescribed for administration may depend on the severity of disease, with more severe disease or illness needing higher and/or more frequent dosage amounts. The following examples are offered by way of illustration and not by way of limitation.

### EXAMPLES

### Examples 1 to 5: HCQ Pharmaceutical Formulations for Inhalation Administration

A series of HCQ aerosol formulations were studied, each containing the HCQ free base in a strength ranging from 175 mcg to 850 mcg (such as from—0.38% to 1.24%), an ethanol (EtOH) concentration ranging from 4% to 12%, and an HFA propellant concentration ranging from 86% to about 96%, all (w/w), as summarized in Table 2. Note that Formulation 2 in Examples 1 and 2 were prepared using different methods.

### Example 1 (Formulation 2 in Table 2)

In one embodiment, the formulation contains 0.38% w/w HCQ free base, 5% w/w EtOH, and 94.62% w/w HFA 134a, which was prepared by:
i) Adding 0.63 g mixture of HCQ base and EtOH (1:13.146 w/w ratio) into an aerosol canister and crimping a 50 μL valve onto it. HCQ base solution is from NaOH method.
ii) Pressurized filling about 11.07 g HFA 134a into the canister and mixing well.
iii) Cascade impactor tests showed that fine particle mass (FPM) (Stage 3-filter) is 161.5 μg (92.3%) and extra-fine particle mass (EPM) (Stage 6-filter) is 86.8 μg (49.6%) per actuation.

### Example 2 (Formulation 2 in Table 2)

In one embodiment, the formulation contains 0.38% w/w HCQ free base, 5% w/w EtOH, and 94.62% w/w HFA 134a, which was prepared by:
i) Adding 44.5 mg HCQ base from EtOAc method and 0.585 g anhydrous ethanol into an aerosol canister and crimping a 50 μL valve onto it.
ii) Pressurized filling ˜11.07 g HFA 134a into the canister and mixing well.
iii) Cascade impactor tests showed that FPM (Stage 3-filter) 168.3 μg (96.2% delivery rate) and EPM (Stage 6-filter) is 94.3 μg (53.9% delivery rate) per actuation.

### Example 3 (Formulation 10 in Table 2)

In one embodiment, the formulation contains 0.443% w/w HCQ free base, 5.5% w/w EtOH and 94.057% w/w HFA 134a, which was prepared by:
i) Adding 51.8 mg HCQ base from EtOAc method and 0.644 g anhydrous ethanol into an aerosol canister and crimping a 50 μL valve onto it.
ii) Pressurized filling ˜11.0 g HFA 134a into the canister and mixing well.
iii) Cascade impactor tests showed that FPM (Stage 3-filter) 199.1 μg (97.1%) and EPM (Stage 6-filter) is 94.3 μg (46.0%) per actuation.

### Example 4 (Formulation 11 in Table 2)

In one embodiment, the formulation contains 0.620% w/w HCQ free base, 6.0% w/w EtOH and 93.36% w/w HFA 134a, which is prepared by:
i) Adding 72.5 mg HCQ base from EtOAc method and 0.703 g anhydrous ethanol into an aerosol canister and crimping a 50 μL valve onto it.
ii) Pressurized filling ˜11.0 g HFA 134a into the canister and mixing well.
iii) Cascade impactor tests showed that FPM (Stage 3-filter) is 340.4 μg (85.1%) and EPM (Stage 6-filter) is 135.3 μg (33.8%) per actuation

### Example 5 (Formulation 12 in Table 2)

In one embodiment, the formulation contains 1.242% w/w HCQ free base, 7% w/w EtOH and 91.558% w/w HFA 227, which is prepared by:
i) Adding 145.2 mg HCQ base from EtOAc method and 0.820 g anhydrous ethanol into an aerosol canister and crimping a 50 μL valve onto it.
ii) Pressurized filling ˜11.0 g HFA 227 into the canister and mixing well.
iii) Cascade impactor tests showed that FPM (Stage 3-filter) is 625.2 μg (73.6%) and EPM (Stage 6-filter) is 135.3 μg (15.9%) per actuation

TABLE 2
HCQ Pharmaceutical Formulations 1-12

| Formulation | HCQ-base | EtOH | HFA 134a | Surfactant | API |
|---|---|---|---|---|---|
| No. | % w/w | % w/w | % w/w | % w/w | Strength |
| 1 | 0.380% | 4.000% | 95.620% | — | 175 mcg |
| 2 | 0.380% | 5.000% | 94.620% | — | 175 mcg |
| 3 | 0.540% | 5.000% | 94.460% | — | 250 mcg |
| 4 | 1.080% | 12.000% | 86.920% | — | 500 mcg |
| 5 | 0.430% | 5.000% | 94.570% | — | 200 mcg |
| 6 | 0.600% | 6.000% | 93.400% | — | 275 mcg |
| 7 | 0.760% | 8.000% | 91.240% | — | 350 mcg |
| 8 | 0.443% | 4.500% | 95.057% | — | 205 mcg |
| 9 | 0.443% | 5.000% | 94.557% | — | 205 mcg |
| 10 | 0.443% | 5.500% | 94.057% | — | 205 mcg |
| 11 | 0.620% | 6.000% | 93.360% | Poloxamer | 400 mcg |
| | | | | 124, | |
| | | | | 0.02% | |
| 12 | 1.242% | 7.000% | HFA 227, | Poloxamer | 850 mcg |
| | | | 91.558% | 182, 0.2% | |

In Table 2 above, Formulations 1-12 are examples of embodiments of the disclosed HCQ pharmaceutical formulations for treating a pulmonary disease, such as COVID-19. In particular, as shown further in Table 7, Formulation 11 advantageously provided the most effective results when considering both drug delivery rate (such as, percentage by weight of extra-fine particle mass (EPM) and fine particle mass (FPM) by weight) and drug delivery amount (such as, dosage quantity) to a patient's upper respiratory tract and a deep portion of the lung where the alveoli are located.

### Example 6: MDI Actuator Orifice Size Selection for Controlling Particle Size Distribution

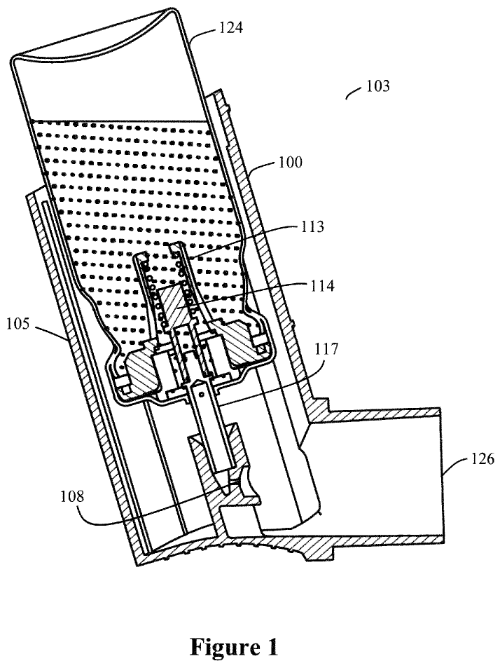

FIG. 1 shows an example MDI actuator 100 for an example MDI 103, which has a body 105 and a cap (not shown in FIG. 1), which may cover the mouthpiece 126 of MDI actuator 100 and provide protection of the mouthpiece 126. MDI actuator 100 accommodates individual stand-alone use such that a patient may grasp the MDI actuator and proceed to actuate the MDI to dispense any of the medications described in the Examples of the present disclosure (referred to herein collectively as “the medication”) as an aerosolized formulation. In addition, MDI 103 includes a canister 124 and a stem 117. The canister 124 may be a pressurized aluminum, or some other metal, canister capable of storing any of the medications described in the Examples. The canister 124 functions to dispense, per actuation (e.g., per spray) using MDI actuator 100, a metered-dose of the medication, which additionally includes a propellant, such as 1,1,1,2-Tetrafluoroethane (HFA 134a) and other ingredients as described by the Examples. In this way, the MDI actuator is suitable for the treatment or prophylaxis of a pulmonary disease, such as COVID-19. In some embodiments, the medication contained within the canister 124 includes an anti-viral therapeutic agent, such as hydroxychloroquine (HCQ), in a free base thereof, or the pharmaceutically acceptable salts thereof. The anti-viral therapeutic agent is delivered by the MDI actuator 100 throughout a respiratory tract of a patient, including the upper and lower respiratory tract, and peripheral, deep lungs where alveoli are located.

As shown in FIG. 1, MDI 103 is aligned within the body 105 to output a spray, using MDI actuator 100 of a metered dose of an API (such as a formulation including HCQ) within MDI 103. More particularly, the body 105 aligns the stem 117 of the MDI 103 with various functional mechanical components inside of MDI actuator 100 that are used to actuate dispensation of a medication (such as those described in the Examples above) from the MDI 103. FIG. 1 is a cross-sectional view of the MDI actuator 100 and shows various functional and mechanical components inside of MDI actuator 100. Such components are well-known and therefore are not described in further detail herein. Briefly, MDI actuator 100 includes orifice 108, mouthpiece 126, stem 117, spring 113, and buffer 114. The medication is dispensed, as an actuation (or spray), out of the orifice 108, through mouthpiece 126, into a patient's mouth. In this way, the medication travels through the respiratory tract of the patient for targeted delivery, such as toward the deep and/or peripheral lungs. More particularly, as described further herein, the orifice 108 has a function of controlling drug particle delivery characteristics of the antiviral formulation. For example, the orifice 108 can have a diameter of a specific size, such as 0.20 mm. The diameter size of orifice 108 controls the proportion of particles having a specific size that can be dispersed outwardly from the MDI actuator 100. That is, size of the orifice 108 directs how much of a dose includes particles of a specific size. Accordingly, size of the orifice 108 controls the particle size distribution of the medication dispersed from the MDI 103 such that particles of a certain size proceed to contact corresponding locations of infection lesions within the respiratory tract.

In further detail, the orifice 108 of the MDI actuator 100 shown in FIG. 1 may be sized with an orifice having a diameter of about or exactly 0.20 mm to dispense fine API particle sizes, such as API particles having a diameter of about 1.1 μm or less. Alternatively, in some embodiments, orifice 108 may be sized to have a diameter at about or exactly 0.42 mm, or at about or exactly 0.28 mm. In some embodiments, actuator 100 is made of, or made substantially of, polyoxymethylene (POM), polypropylene (PP), polycarbonate (PC), acrylonitrile butadiene styrene (ABS), high-density polyethylene (HDPE), or other suitable materials. In other embodiments, actuator 100 can be made of, or made substantially of, clear or transparent PC, or other suitable materials to enable viewing of an add-on dose-counter.

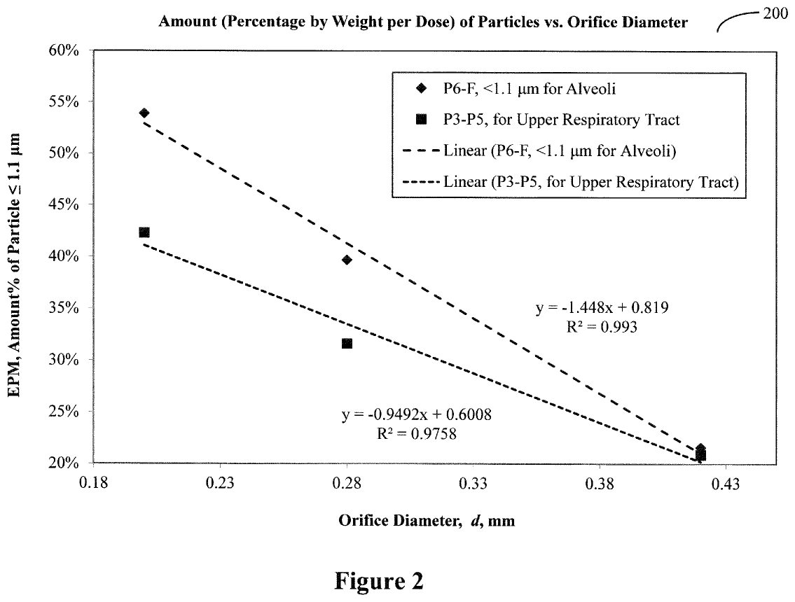

As described above, the disclosed MDI actuator orifice may include a diameter that is particularly sized to correspondingly control dispensation of the aerosolized medication to include a defined fraction of fine or extra-fine particles per dosage, such as particles having a diameter of about 1.1 μm or less. Table 4, below, outlines a study of the amount percentage (%) of fine API particles having particle diameters of less than about 1.1 μm versus orifice diameter size of the disclosed MDI actuators, for example MDI actuator 100. In addition, observed results are depicted in a graph 200 of FIG. 2. More specifically, the graph of FIG. 2 shows the MDI actuator 100 tested with multiple different diameter sizes of the orifice. In particular, as shown in Table 3 below, 3 different MDI actuators were tested: (1) MDI actuator having 0.42 mm orifice diameter (MDI Actuator A), (2) MDI actuator having 0.28 mm orifice diameter (MDI Actuator B), and (3) MDI actuator having 0.20 mm orifice diameter (MDI Actuator C).

TABLE 3
HCQ Actuators

| Actuators | A | B | C |
|---|---|---|---|
| Orifica Size (mm) | 0.42 | 0.28 | 0.20 |

MDI Actuator A, MDI Actuator B, and MDI Actuator C were evaluated using Formulation 2 described in the Examples, which has a strength of 0.175 mg (or 175 mcg) of HCQ, provided as HCQ free base at a concentration level of 0.38% (w/w), with 5.0% ethanol alcohol (w/w), 94.62% propellant HFA 134a (w/w) (“w/w” denotes weight by weight).

Items shown in Table 4 below represent different cascade impactor measured particle size distributions (in μm) of aerosolized formulations intended for delivery into a patient's respiratory tract. For examples, the alveoli are primarily located in regions of the respiratory tract that correspond with at least Stage 6 of a cascade impactor. As a result, particles having particle diameters of 0.65 μm to 1.1 μm are expected to reach the alveoli. That is, the medication described in the Examples is dispersed as an aerosolized formulation upon actuation of the MDI actuator 100 of FIG. 1. The precise size of the orifice 108 used with the MDI actuator 100 influences the particle size distribution of the aerosolized formulation. For example, as shown in FIG. 2, an orifice diameter of 0.20 mm corresponds with an extra-fine particle mass (EPM) of 53.9% (w/w), which presents a marked increase in EPM, relative to other orifice diameters. EPM is defined as the mass of particles having particle diameters less than 1.1 μm compared to the entire mass of a single dose of the medication, e.g., such as those described by the above Examples.

“EPM (Stage 6-filter),” as referred to below in Table 4, represents the total amount and delivery efficiency rate, per actuation of the MDI 103 of FIG. 1, of HCQ particles included in the aerosolized medication having diameters of less than about 1.1 μm. The delivery efficiency rate was determined by: dividing (i) a total amount, per actuation, of HCQ particles having a diameter of less than about 1.1 μm, by (ii) an expected API metered dose per actuation. In the tests outlined in Table 4, the expected API metered dose per actuation was 175 mcg, and the EPM is the total amount, per actuation, of HCQ particles having a diameter of less than about 1.1 μm.

TABLE 4
Formulation 2 with MDI Actuator A, MDI Actuator B, and MDI Actuator C

| | | Formulation 2, | | Formulation 2, | | Formulation 2, | |
|---|---|---|---|---|---|---|---|
| | | Actuator A | | Actuator B | | Actuator C | |
| | | 175 mcg | | 175 mcg | | 175 mcg | |
| Items | | | Compared | | Compared | | Compared |
| | Aerosol | Mass/Spray | to | Mass/Spray | to | Mass/Spray | to |
| Stage # | Size | (mcg) | strength | (mcg) | strength | (mcg) | strength |
| Throat | μm | 82.3 | 47.0% | 24.3 | 13.9% | 11.8 | 6.8% |
| 0 | >9.0 | 1.4 | 0.8% | 0.6 | 0.3% | 0.4 | 0.2% |
| 1 | 5.8-9 | 0.8 | 0.4% | 0.6 | 0.3% | 0.5 | 0.3% |
| 2 | 4.7-5.8 | 1.5 | 0.8% | 1.3 | 0.7% | 0.8 | 0.5% |
| 3 | 3.3-4.7 | 8.6 | 4.9% | 7.7 | 4.4% | 6.5 | 3.7% |
| 4 | 2.1-3.3 | 13.7 | 7.8% | 19.4 | 11.1% | 25.7 | 14.7% |
| 5 | 1.1-2.1 | 14.2 | 8.1% | 28.2 | 16.1% | 41.7 | 23.9% |
| 6 | 0.65-1.1 | 4.4 | 2.5% | 13.4 | 7.7% | 23.2 | 13.3% |
| 7 | 0.43- | 2.0 | 1.2% | 4.7 | 2.7% | 6.5 | 3.7% |
| | 0.65 | | | | | | |
| Filter | <0.43 | 31.5 | 18.0% | 51.3 | 29.3% | 64.6 | 36.9% |

| | | | | | | |
|---|---|---|---|---|---|---|
| Total | 160.3 | 91.6% | 151.6 | 86.6% | 181.9 | 103.9% |
| % Recovery | 92% | NA | 87% | NA | 104% | NA |
| (85%~115%) | | | | | | |
| Stage3~5 | 36.5 | 20.9% | 55.4 | 31.6% | 74.0 | 42.3% |
| CPM (Throat-2) | 86.0 | 49.1% | 26.8 | 15.3% | 13.6 | 7.8% |
| EPM (Stage 6-filter) | 37.9 | 21.6% | 69.4 | 39.7% | 94.3 | 53.9% |
| ISM (Stage 0-filter) | 78.0 | 44.6% | 127.2 | 72.7% | 170.1 | 97.2% |
| FPM (Stage 3-filter) | 74.4 | 42.5% | 124.8 | 71.3% | 168.3 | 96.2% |

In Table 4 shown above (as well as for other similar Tables of the present disclosure): “Mass/Spray” is the mass of particles collected at each indicated Stage during testing with a cascade impactor; Total” is the total particle mass delivered per each actuation. “% Recovery” is the total particle mass compared to theoretical mass of 175 mg per each actuation, where about 85% to 115% is within the specification of the cascade impactor; “CPM” is “Coarse Particle Mass”, which is particle mass collected on throat, Stage 1 and Stage 2; “EPM” is “Extra-fine Particle Mass”, which is particle mass collected on Stage 6,7, and beyond (Stage 6-filter); “ISM” is “Impactor-Sized Mass”, which is particle mass collected on stage 0 through filter (Stage 0-filter); “FPM” is “Fine Particle Mass”, which is particle mass collected on Stage 3 to Stage 7 and beyond (Stage 3-filter).

Table 5 below shows that using MDI Actuator C (orifice 0.20 mm), the delivery rate of Stage 3 to 5 and Stage 6 to filter (such as Stage 6 to Stage 7 and beyond) is 42% and 54%, respectively, both of which are significantly higher than the delivery rate using MDI Actuator A (with an orifice diameter of 0.42 mm), and the one with actuator B (0.28 mm). Table 4 and graph 200 of FIG. 2, demonstrate the relationship between the actuator orifice size and the delivery rate as “Amount % of Particles” shown in FIG. 2, also defined as the percentage, by weight, of extra-fine particle (EPM) mass per dosage. Observed results indicate that the delivery rate is approximately linear with actuator orifice size.

As shown in Table 5, MDI Actuator C, which had an orifice diameter of about 0.20 mm, provided the strongest results in terms of delivery efficiency rate, as compared to MDI Actuator A or MDI Actuator B. In particular, MDI Actuator C provided a delivery efficiency rate of about 53.9% (w/w) for “Stage 6-Filter, <1.1 μm for Alveoli,” meaning that about 53.9% (w/w) of the HCQ particles dispensed, per actuation, by MDI Actuator C had particle diameters of less than 1.1 μm. This particle size is advantageous in delivering HCQ to a portion of the lungs (such as, deep-lung and/or alveoli) that correspond to Stage 6 to Stage 7 and beyond as measured by a cascade impactor. As a result, particles having diameters of less than 1.1 μm can effectively treat pulmonary diseases, such as COVID-19, within the alveoli. In this way, with respect to “Stage 6-Filter, <1.1 μm for Alveoli,” MDI Actuator C, a delivery efficiency rate of 53.9% (w/w) represents a significant improvement of MDI Actuators A and B having delivery rates of 21.6% and 39.7%, respectively. Accordingly, MDI Actuator C was selected for HCQ delivery.

TABLE 5
Assessment of Amount of Small Particles
That Can Reach Alveoli for HCQ MDI

| MDI Actuators | A | B | C |
|---|---|---|---|
| Orifice Size (mm) | 0.42 | 0.28 | 0.20 |
| Amount % of Particle | | | |
| Stage 6-Filter, <1.1 μm for Alveoli | 21.6% | 39.7% | 53.9% |
| Stage 3~5, for Upper Respiratory | 20.9% | 31.6% | 42.3% |
| Tract | | | |

### Example 7: Different HCQ Formulations with MDI Actuators

Initially, several formulations with API strengths from 175 mcg to 500 mcg and with an ethanol concentration from 4% to 12% (w/w) were evaluated by cascade impactor tests using MDI Actuator A (0.42 mm). With higher strength, a higher concentration of ethanol is necessary for dissolving the API completely within the formulation. Results showed that with higher strength of 500 mcg HCQ in 12% w/w ethanol (Formulation 4), the delivery rates of Stage 3-5 and Stage 6-filter are about 9% and 10%, respectively, which are relatively low. The lower strength of 175 mcg (Formulation 1 and 2) with 4% to 5% (w/w) ethanol, the delivery rates of Stage 3-5 and Stage 6-filter are about 21% to 23% and 21% to 22%, respectively, which are comparatively better than indicated for high API strength in high ethanol concentration. This finding indicates that low ethanol concentration in the formulation is necessary for the formation of smaller particles and for higher a delivery rate to the deep lung. However, when both high strength and a high delivery rate are required for the formulation, the solubility of HCQ in ethanol/HFA 134a limited the formulation strength. Therefore, Formulation 11 as shown in Table 7 was developed to meet both requirements. “Compared to strength” is calculated by dividing the observed or recovered mass/spray by the total API loading level per dose. For example, the strength (dosage) of 175 mcg of Formulation 1, Actuator A for a region of the lung corresponding to that measured by Stage 3 on a cascade impactor, 7.9 mcg was observed or recovered (Mass/Spray), which, when divided by the strength of 175 mcg, yields a “compared to strength” of 4.5%.

TABLE 6
Different Formulations with MDI Actuator A

| | | Formulation 1, | | Formulation 2, | | Formulation 3, | | Formulation 4, | |
|---|---|---|---|---|---|---|---|---|---|
| | | Actuator A | | Actuator A | | Actuator A | | Actuator A | |
| | | 175 mcg | | 175 mcg | | 250 mcg | | 500 mcg | |
| Items | | Mass/ | Compared | Mass/ | Compared | Mass/ | Compared | Mass/ | Compared |
| | Aerosol | Spray | to | Spray | to | Spray | to | Spray | to |
| Stage # | Size | (mcg) | strength | (mcg) | strength | (mcg) | strength | (mcg) | strength |
| Throat | μm | 84.1 | 48.1% | 82.3 | 47.0% | 126.2 | 50.5% | 339.1 | 67.8% |
| 0 | >9.0 | 0.9 | 0.5% | 1.4 | 0.8% | 2.3 | 0.9% | 10.1 | 2.0% |
| 1 | 5.8-9 | 0.6 | 0.4% | 0.8 | 0.4% | 1.5 | 0.6% | 2.9 | 0.6% |
| 2 | 4.7-5.8 | 1.1 | 0.7% | 1.5 | 0.8% | 3.0 | 1.2% | 6.4 | 1.3% |
| 3 | 3.3-4.7 | 7.9 | 4.5% | 8.6 | 4.9% | 15.3 | 6.1% | 18.2 | 3.6% |
| 4 | 2.1-3.3 | 14.7 | 8.4% | 13.7 | 7.8% | 19.4 | 7.8% | 16.2 | 3.2% |
| 5 | 1.1-2.1 | 17.5 | 10.0% | 14.2 | 8.1% | 17.7 | 7.1% | 10.6 | 2.1% |
| 6 | 0.65-1.1 | 5.6 | 3.2% | 4.4 | 2.5% | 7.0 | 2.8% | 2.9 | 0.6% |
| 7 | 0.43-0.65 | 2.1 | 1.2% | 2.0 | 1.2% | 2.9 | 1.1% | 2.4 | 0.5% |
| Filter | <0.43 | 28.5 | 16.3% | 31.5 | 18.0% | 30.6 | 12.2% | 43.8 | 8.8% |

| | | | | | | | | |
|---|---|---|---|---|---|---|---|---|
| Total | 163.1 | 93.2% | 160.3 | 91.6% | 225.8 | 90.3% | 452.7 | 90.5% |
| % Recovery (85%~115%) | 93% | NA | 92% | NA | 90% | NA | 91% | NA |
| Stage 3~5 | 40.1 | 22.9% | 36.5 | 20.9% | 52.3 | 20.9% | 45.0 | 9.0% |
| CPM (Adaptor/Throat-2) | 86.8 | 49.6% | 86.0 | 49.1% | 133.0 | 53.2% | 358.5 | 71.7% |
| EPM (Stage 6-filter) | 36.2 | 20.7% | 37.9 | 21.6% | 40.5 | 16.2% | 49.1 | 9.8% |
| ISM (Stage 0-filter) | 78.9 | 45.1% | 78.0 | 44.6% | 99.6 | 39.8% | 113.5 | 22.7% |
| FPM (Stage 3-filter) | 76.3 | 43.6% | 74.4 | 42.5% | 92.8 | 37.1% | 94.1 | 18.8% |

After selection of an appropriate actuator orifice size, more formulations with strength from 175 mcg to 850 mcg and with ethanol concentration from 4 to 12% (w/w) were studied by using MIDI Actuator C. By comparison both the delivery rate on Stage 3-5 and Stage 6-filter of these formulations in Tables 2 and 7, as well as the bar charts, it indicated that Formulation 11 (400 mcg strength, 6% EtOH) would be comparatively most efficient when considering drug amount and delivery rate of HCQ to the lung.

TABLE 7
Different Formulations with Actuator C

| | | Formulation 2, | | Formulation 5, | | Formulation 6, | | Formulation 7, | | Formulation 8, | |
|---|---|---|---|---|---|---|---|---|---|---|---|
| | | Actuator C | | Actuator C | | Actuator C | | Actuator C | | Actuator C | |
| | | 175 mcg | | 200 mcg | | 275 mcg | | 350 mcg | | 205 mcg | |
| Items | | Mass/ | Compare | Mass/ | Compare | Mass/ | Compare | Mass/ | Compare | Mass/ | Compare |
| | Aerosol | Spray | to | Spray | to | Spray | to | Spray | to | Spray | to |
| Stage# | Size | (mcg) | strength | (mcg) | strength | (mcg) | strength | (mcg) | strength | (mcg) | strength |
| Throat | μm | 11.8 | 6.8% | 11.1 | 5.6% | 20 | 7.3% | 39.8 | 11.4% | 14.8 | 7.2% |
| 0 | >9.0 | 0.4 | 0.2% | 0.5 | 0.2% | 0.8 | 0.3% | 1.3 | 0.4% | 0.8 | 0.4% |
| 1 | 5.8-9 | 0.5 | 0.3% | 0.7 | 0.3% | 1 | 0.4% | 1.7 | 0.5% | 1.2 | 0.6% |
| 2 | 4.7-5.8 | 0.8 | 0.5% | 1.5 | 0.7% | 2.5 | 0.9% | 3.8 | 1.1% | 2.1 | 1.0% |
| 3 | 3.3-4.7 | 6.5 | 3.7% | 8.1 | 4.0% | 14.7 | 5.4% | 26.1 | 7.5% | 12.3 | 6.0% |
| 4 | 2.1-3.3 | 25.7 | 14.7% | 30.1 | 15.0% | 53.3 | 19.4% | 72.9 | 20.8% | 42.6 | 20.8% |
| 5 | 1.1-2.1 | 41.7 | 23.9% | 59.9 | 30.0% | 74.8 | 27.2% | 89.4 | 25.5% | 64.4 | 31.4% |
| 6 | 0.65-1.1 | 23.2 | 13.3% | 24.6 | 12.3% | 29.1 | 10.6% | 29.2 | 8.3% | 26.9 | 13.1% |
| 7 | 0.43-0.65 | 6.5 | 3.7% | 7.8 | 3.9% | 8.2 | 3.0% | 8.8 | 2.5% | 8.2 | 4.0% |
| Filter | <0.43 | 64.6 | 36.9% | 67.1 | 33.5% | 56.3 | 20.5% | 89.7 | 25.6% | 65 | 31.7% |
| Total | | 181.9 | 103.9% | 211.3 | 105.6% | 260.8 | 94.8% | 362.7 | 103.6% | 238.1 | 116.2% |
| % Recovery | | 104% | NA | 106% | NA | 95% | NA | 104% | NA | 116% | NA |
| (85%~115%) | | | | | | | | | | | |
| Stage 3-5 | | 74 | 42.3% | 98.1 | 49.0% | 142.8 | 51.9% | 188.4 | 53.8% | 119.3 | 58.2% |
| CPM | | 13.6 | 7.8% | 13.8 | 6.9% | 24.3 | 8.8% | 46.6 | 13.3% | 18.8 | 9.2% |
| (Adaptor/Throat-2) | | | | | | | | | | | |
| EPM (Stage 6- [symbol] | | 94.3 | 53.9% | 99.4 | 49.7% | 93.7 | 34.1% | 128 | 36.5% | 100 | 48.8% |
| ISM (Stage 0-filter) | | 170.1 | 97.2% | 200.1 | 100.1% | 240.8 | 87.6% | 322.9 | 92.3% | 223.4 | 109.0% |
| FPM (Stage 3-filter) | | 168.3 | 96.2% | 197.5 | 98.7% | 236.5 | 86.0% | 316.1 | 90.3% | 219.4 | 107.0% |
| | | | | Formulation 9, | | Formulation 10, | | Formulation 11, | | Formulation 12, | |
| | | | | Actuator C | | Actuator C | | Actuator C | | Actuator C | |
| | | | | 205 mcg | | 205 mcg | | 400 mcg | | 850 mcg | |
| | | Items | | Mass/ | Compare | Mass/ | Compare | Mass/ | Compare | Mass/ | Compare |
| | | | Aerosol | Spray | to | Spray | to | Spray | to | Spray | to |
| | | Stage# | Size | (mcg) | strength | (mcg) | strength | (mcg) | strength | (mcg) | strength |
| | | Throat | μm | 15.4 | 7.5% | 17.7 | 8.6% | 55.6 | 13.9% | 158.9 | 18.7% |
| | | 0 | >9.0 | 0.6 | 0.3% | 0.6 | 0.3% | 1.3 | 0.3% | 16.6 | 2.0% |
| | | 1 | 5.8-9 | 0.6 | 0.3% | 1.2 | 0.6% | 1.6 | 0.4% | 30.5 | 3.6% |
| | | 2 | 4.7-5.8 | 1.6 | 0.8% | 1.6 | 0.8% | 4.5 | 1.1% | 60.9 | 7.2% |
| | | 3 | 3.3-4.7 | 9.6 | 4.7% | 9.5 | 4.6% | 29.5 | 7.4% | 181.0 | 21.3% |
| | | 4 | 2.1-3.3 | 33.8 | 16.5% | 36.3 | 17.7% | 72.6 | 18.1% | 182.0 | 21.4% |
| | | 5 | 1.1-2.1 | 64.8 | 31.6% | 59 | 28.8% | 103.1 | 25.8% | 126.9 | 14.9% |
| | | 6 | 0.65-1.1 | 24.9 | 12.2% | 21.7 | 10.6% | 40.2 | 10.0% | 35.0 | 4.1% |
| | | 7 | 0.43-0.65 | 7.8 | 3.8% | 7.3 | 3.6% | 11.7 | 2.9% | 14.8 | 1.7% |
| | | Filter | <0.43 | 65.4 | 31.9% | 65.3 | 31.8% | 83.3 | 20.8% | 85.5 | 10.1% |

| | | | | | | | | |
|---|---|---|---|---|---|---|---|---|
| Total % | 224.6 | 109.5% | 220.2 | 107.4% | 403.3 | 100.8% | 892.2 | 105.0% |
| Recovery | 110% | NA | 107% | NA | 101% | NA | 105.0% | NA |
| (85%~115%) | | | | | | | | |
| Stage 3-5 | 108.2 | 52.8% | 104.8 | 51.1% | 205.1 | 51.3% | 489.9 | 57.6% |
| CPM | 18.2 | 8.9% | 21.1 | 10.3% | 62.9 | 15.7% | 266.9 | 31.4% |
| (Adaptor/Throat-2) | | | | | | | | |
| EPM (Stage 6- [symbol] | 98.2 | 47.9% | 94.3 | 46.0% | 135.3 | 33.8% | 135.3 | 15.9% |
| ISM (Stage 0-filter) | 209.2 | 102.0% | 202.5 | 98.8% | 347.8 | 86.9% | 733.3 | 86.3% |
| FPM (Stage 3-filter) | 206.4 | 100.7% | 199.1 | 97.1% | 340.4 | 85.1% | 625.2 | 73.6% |

[symbol]

indicates data missing or illegible when filed

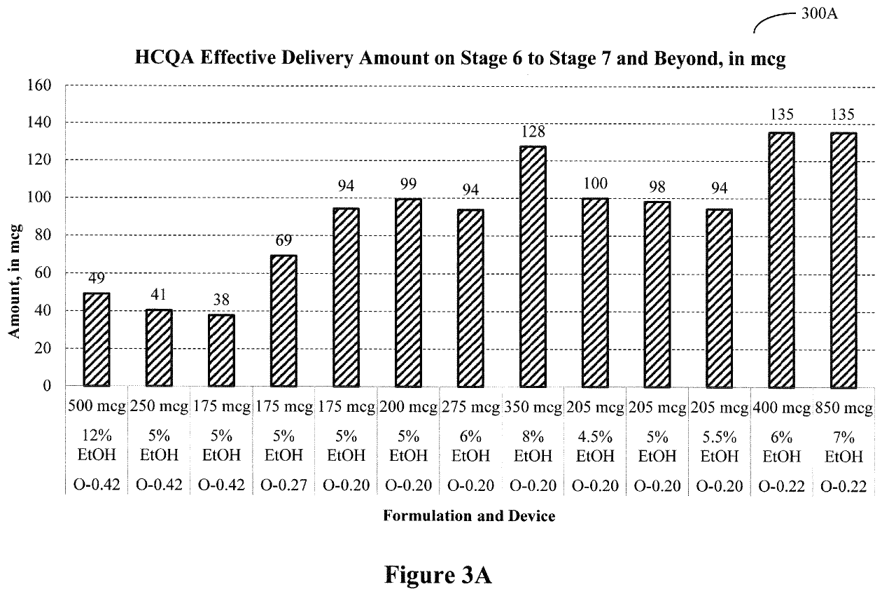

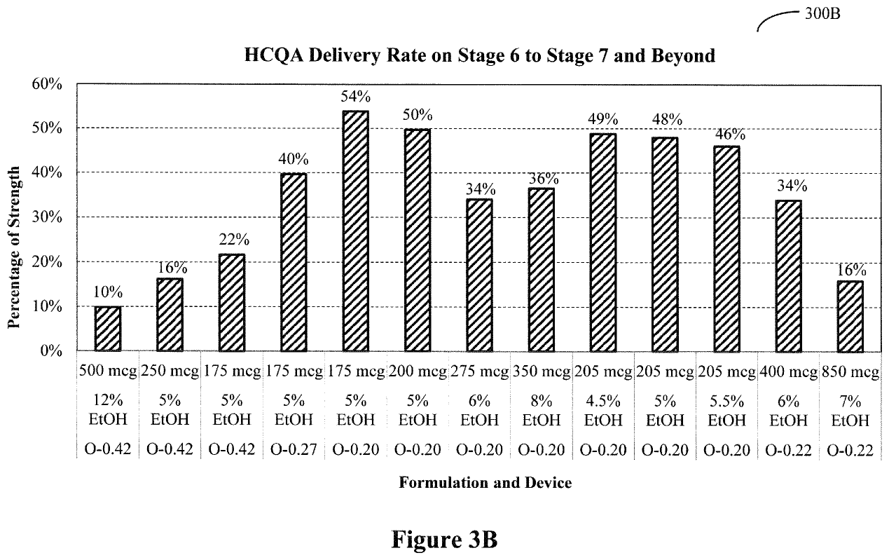

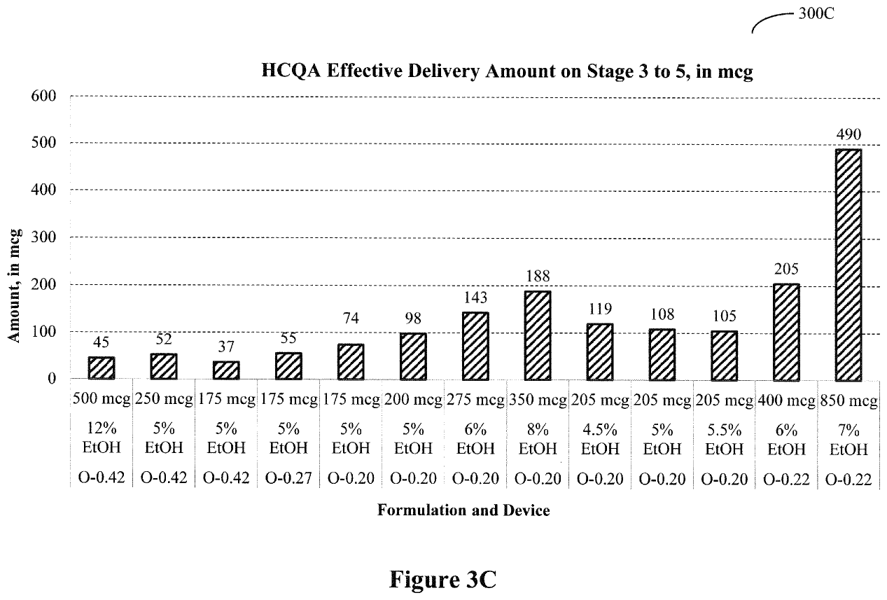

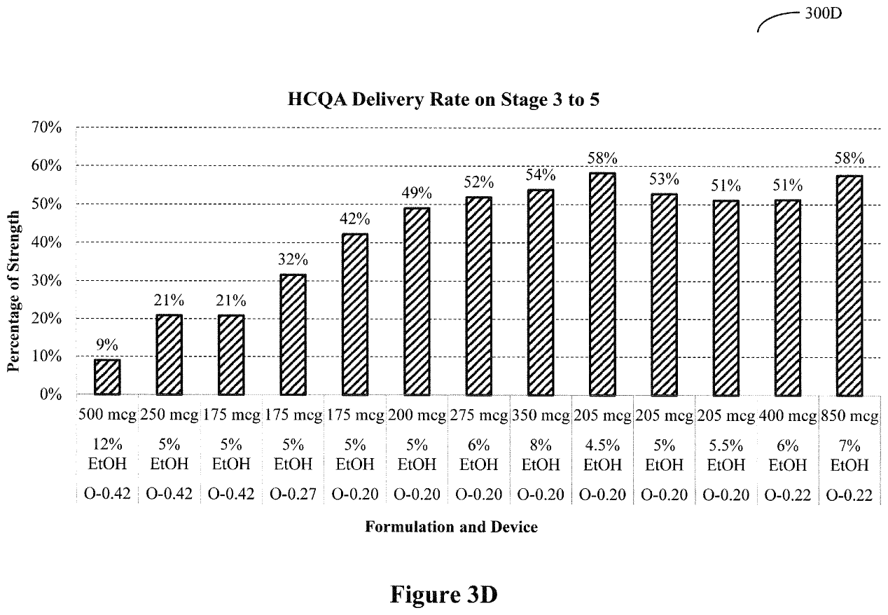

Tables 8 and 9, as well as bar charts 300A-300D, shown in FIGS. 3A-3D, respectively, show HCQ delivery amount and delivery rate on stage 6-filter and Stage 3-5, respectively, in different formulations (API strength from 175 mcg to 850 mcg and ethanol concentration from 4.5% to 12% w/w) and different actuators for stand-alone use (e.g., the orifice 108 having a discrete a diameter size selected within a range from 0.20 mm to 0.42 mm). The results shown in Table 8, below, demonstrate that Formulation 11 with MDI Actuator C was able to deliver the most HCQ, with an API strength of 400 mcg, to Stage 6-filter when measured in a cascade impactor and was therefore a viable choice for HCQ medication delivery.

TABLE 8
HCQ Delivery Efficiency for Deep Lung (Alveoli) (Stage 6-filter)

| | | | | | Amount of | Deep Lung |
|---|---|---|---|---|---|---|
| | | | | | API | (Alveoli) |
| | | | | API | Delivered | API |
| Formulation | | | | Strength | <1.1 mcm | Delivery |
| No. | Formulation and | | API Strength | Per 1 | Per 1 | Rate Per 1 |
| (From | Device | | Per 1 Metered | Metered | Metered | Metered |
| Table 1) | Actuator | EtOH | Dose | Dose | Dose | Dose |
| 4 | O-0.42 | 12% EtOH | 500 mcg | 500 | 49.1 | 9.8% |
| 3 | O-0.42 | 5% EtOH | 250 mcg | 250 | 40.5 | 16.2% |
| 2 | O-0.42 | 5% EtOH | 175 mcg | 175 | 37.9 | 21.7% |
| 2 | O-0.27 | 5% EtOH | 175 mcg | 175 | 69.4 | 39.7% |
| 2 | O-0.20 | 5% EtOH | 175 mcg | 175 | 94.3 | 53.9% |
| 5 | O-0.20 | 5% EtOH | 200 mcg | 200 | 99.4 | 49.7% |
| 6 | O-0.20 | 6% EtOH | 275 mcg | 275 | 93.7 | 34.1% |
| 7 | O-0.20 | 8% EtOH | 350 mcg | 350 | 127.7 | 36.5% |
| 8 | O-0.20 | 4.5% EtOH | 205 mcg | 205 | 100 | 48.8% |
| 9 | O-0.20 | 5% EtOH | 205 mcg | 205 | 98.2 | 47.9% |
| 10 | O-0.20 | 5.5% EtOH | 205 mcg | 205 | 94.3 | 46.0% |
| 11 | O-0.22 | 6% EtOH | 400 mcg | 400 | 135.3 | 33.8% |
| 12 | O-0.22 | 7% EtOH | 850 mcg | 850 | 135.3 | 15.9% |

TABLE 9
HCQ Aerosol (“HCQA”) Delivery Efficiency for the Upper Respiratory
Tract (corresponding to Stages 3-5 in a Cascade Impactor)

| | | | | | | Upper |
|---|---|---|---|---|---|---|
| | | | | | | Respiratory |
| | | | API | API | Amount API | Tract API |
| Formulation | | | Strength | Strength | Delivered | Delivery |
| No. | Formulation | | Per 1 | Per 1 | 1.1~5 mcm Per | Rate Per 1 |
| (From | and Device | | Metered | Metered | 1 Metered | Metered |
| Table 1) | Actuator | EtOH | Dose | Dose | Dose | Dose |
| 4 | O-0.42 | 12% EtOH | 500 mcg | 500 | 45 | 9.0% |
| 3 | O-0.42 | 5% EtOH | 250 mcg | 250 | 52.3 | 20.9% |
| 2 | O-0.42 | 5% EtOH | 175 mcg | 175 | 36.5 | 20.9% |
| 2 | O-0.27 | 5% EtOH | 175 mcg | 175 | 55.4 | 31.7% |
| 2 | O-0.20 | 5% EtOH | 175 mcg | 175 | 74 | 42.3% |
| 5 | O-0.20 | 5% EtOH | 200 mcg | 200 | 98.1 | 49.1% |
| 6 | O-0.20 | 6% EtOH | 275 mcg | 275 | 142.8 | 51.9% |
| 7 | O-0.20 | 8% EtOH | 350 mcg | 350 | 188.4 | 53.8% |
| 8 | O-0.20 | 4.5% EtOH | 205 mcg | 205 | 119.3 | 58.2% |
| 9 | O-0.20 | 5% EtOH | 205 mcg | 205 | 108.2 | 52.8% |
| 10 | O-0.20 | 5.5% EtOH | 205 mcg | 205 | 104.8 | 51.1% |
| 11 | O-0.22 | 6% EtOH | 400 mcg | 400 | 205.1 | 51.3% |
| 12 | O-0.22 | 7% EtOH | 850 mcg | 850 | 489.9 | 57.6% |

In addition, bar charts 300A-300D, shown in FIGS. 3A-3D respectively, show various relationships between HCQ Aerosol (HCQA) effective delivery amounts and orifice diameter size, API strength, ethanol content, and/or the like. More specifically, bar chart 300A of FIG. 3A shows the effective delivery amount, in micrograms (mcg), of HCQA for various API concentration levels, ethanol concentration levels, and MDI actuator orifice (such as orifice 108 of FIG. 1) diameter opening sizes. For example, as shown in bar chart 300A, 128 mcg of HCQA was delivered as measured between Stage 6 to filter (Stage 6 to Stage 7 and beyond) in a cascade impactor for an API strength per metered dose of 350 mcg with an ethanol concentration level of 8% and an orifice diameter of 0.20 mm.

Bar chart 300B of FIG. 3B shows the “percentage of strength of HCQA delivery rate” for Stage 6 to filter, where “percentage of strength of HCQA delivery rate” is defined as “compared to strength” earlier. For example, as shown by bar chart 300B of FIG. 3B, for an API strength of 400 mcg, an ethanol concentration level of 6%, and an orifice diameter of 0.22 mm, 34% of the original API strength was observed or recovered from Stage 6 to filter (Stage 6 to Stage 7 and beyond) in a cascade impactor.

Bar chart 300C of FIG. 3C shows the effective delivery amount, in micrograms (“mcg”), of HCQA for various API concentration levels, ethanol concentration levels, and MDI actuator orifice (such as orifice 108 of FIG. 1) diameter opening sizes. For example, as shown in bar chart 300C, 205 mcg of HCQA was delivered as measured between Stages 3 to 5 in a cascade impactor for an API strength per metered dose of 400 mcg with an ethanol concentration level of 6% and an orifice diameter of 0.22 mm.

Bar chart 300D of FIG. 3D shows the “percentage of strength of HCQA delivery rate” for Stage 6 to filter, where “percentage of strength of HCQA delivery rate” is defined as “compared to strength” earlier. For example, as shown by bar chart 300D of FIG. 3D, for an API strength of 400 mcg, an ethanol concentration level of 6%, and an orifice diameter of 0.22 mm, 51% of the original API strength was observed or recovered between Stages 3 to 5 in a cascade impactor.

### Examples 8 to 12: Lung Delivery of Inhaled HCQ in Nonclinical Animal Model

The nonclinical animal study in Examples 8-12 was performed with a breathing tank 400 shown in FIG. 4. The breathing tank 400 is a specially designed stainless steel breathing tank, which is used for mice to breathe in aerosolized formulations of the medications described earlier in the Examples. At the start of each treatment session, a specific number of sprays of a medication formulation were sprayed into the pre-cleaned tank. To ensure consistent concentration of test article, a stirring fan installed inside the tank was started at a speed of 400 rpm before the first spray of the test articles (such as, the medications). Thirty seconds after the last spray, eight mice were mounted to the inhalation chamber to breathe in aerosolized formulations of the medications from the breathing tank for a specified duration. Animals were then taken off the breathing tank (defined as the “0” time point post dosing).

### Example 8: Basic Lung Delivery Characteristics of Single Dose HCQ Inhalation

The purpose of the study is to evaluate the lung delivery characteristics of single dose HCQ inhalation in a mouse model. Ten sprays of HCQ Formulation 5 were sprayed into the pre-cleaned tank. Thirty seconds after the last spray (t=0 minute), eight mice were mounted to the inhalation chamber to breathe the medication aerosol from the breathing tank for 10 minutes.

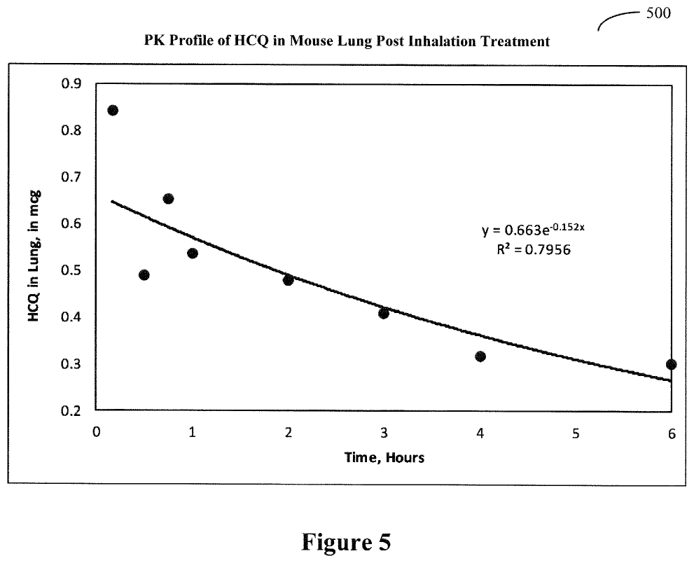

Mice were euthanized and mice lung samples were collected and homogenated at eight time points of 10′, 30′, 45′, 1 h, 2 h, 3 h, 4 h and 6 h after treatment. Four mice were used at each time point and in total 32 mice were studied. All 32 mice appeared healthy and normal after the treatment. Lung HCQ concentration was analyzed using a Liquid Chromatograph Triple Quadrupole Mass Spectrometer (LC/MS/MS) method. The results are summarized in a graph 600 shown in FIG. 5, which shows variation of HCQ concentration with time in mice, and Table 10 below.

TABLE 10
HCQ Amounts Deposited in Mouse Lungs after
HCQ (Formulation 5) Breathing

| Time Points | | Weight, g | | |
|---|---|---|---|---|
| Minutes | Hours | Body | Lung | HCQ in Lungs, μg |
| 10 | 0.17 | 31.0 | 0.188 | 0.84 |
| 30 | 0.5 | 29.5 | 0.176 | 0.49 |
| 45 | 0.75 | 29.1 | 0.183 | 0.65 |
| 60 | 1 | 29.3 | 0.187 | 0.54 |
| 120 | 2 | 29.0 | 0.189 | 0.48 |
| 180 | 3 | 29.3 | 0.158 | 0.41 |
| 240 | 4 | 27.3 | 0.173 | 0.32 |
| 360 | 6 | 28.6 | 0.171 | 0.30 |

As shown in FIG. 5 and above in Table 10, HCQ can quickly reach mouse lungs after inhalation treatment, with the highest lung HCQ deposition amount at the first sampling time (10-minute post dosing). The half-life of HCQ was calculated to be 4.6 hours per the elimination rate constant, Ke=0.152 T⁻¹.

### Example 9: Dose Range and Dose Response Study of HCQ for Lung Delivery

Example 9 was conducted to evaluate the dose response of HCQ for lung delivery. Mice were grouped into three study arms representing low, mid, and high dose levels of the Formulation 11 described in Table 2 and earlier Examples. As shown in Table 11 below, the doses used in the low (4 sprays), mid (8 sprays) and high (16 sprays) levels are 1.6-, 3.2-, and 6.0-times the amount intended in human dose (3 sprays, 0.4 mg HCQ per spray, totally 1.2 mg HCQ per treatment) in the unit of “mg/Kg-body weight” and 3.9-, 8.0- and 15.0-fold high of the intended human use dose in the unit of “mg/Kg-Lung weight”.

TABLE 11
Mouse Treatment Doses

| Respiratory Minute Volume | | 30 mL/min | | | Mouse representative Body weight, w ^{[1]} | | | 30 g | |
|---|---|---|---|---|---|---|---|---|---|
| (RMV), v, ^{[1]} | | | | | | | | | |
| Mouse Deposit Factor (DF) for | | 0.1 | | | Mouse representative Lung weight, w^{[1]} | | | 0.20 g | |
| inhalation f, ^{[1]} | | | | | | | | | |
| Human Dose per Body weight: | | 0.020 mg/kg-body | | | Human Dose per Lung weight: | | | 1.20 mg/kg-lung | |
| | | | | | Mouse | Relative Mouse Dose, | | Dose Ratio: | |
| | # of Sprays | HCQ in | HCQ (c) in | Breath | Dose, | d = D/w ^{[2]} mg/kg | | Mouse vs. | |
| Dose | per | Chamber | Chamber | Time, t | D = c v t f, | mg/Kg- | mg/kg- | Human | |
| Level | Treatment | mg | mg/L | min | (ng) | body | Lung | Body | Lung |
| Low | 4 | 1.6 | 0.052 | 6 | 944 | 0.031 | 4.72 | 1.6 | 3.9 |
| Mid | 8 | 3.2 | 0.107 | 6 | 1926 | 0.064 | 9.6 | 3.2 | 8.0 |
| High | 16 | 6.4 | 0.201 | 6 | 3611 | 0.120 | 18.1 | 6.0 | 15.0 |

^{[1]}: Values are from J. S. Tepper, et al. Int J Toxicol. 35(4): 376-392, (2016).
^{[2]}: “w” is the body weight or the lung weight.

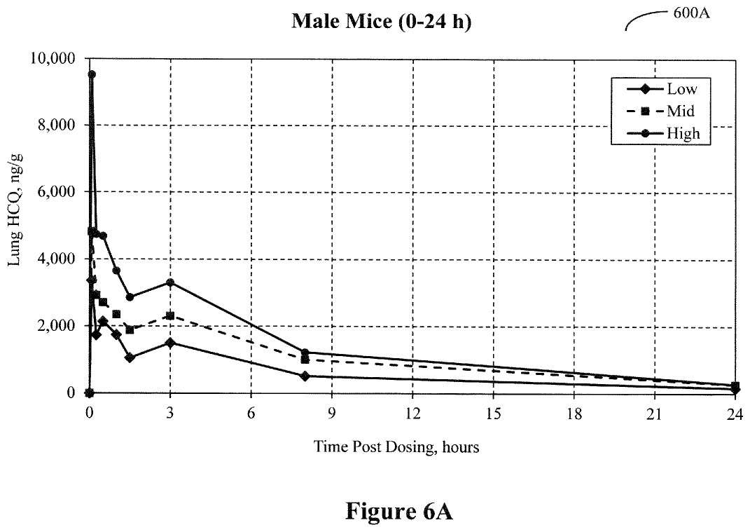

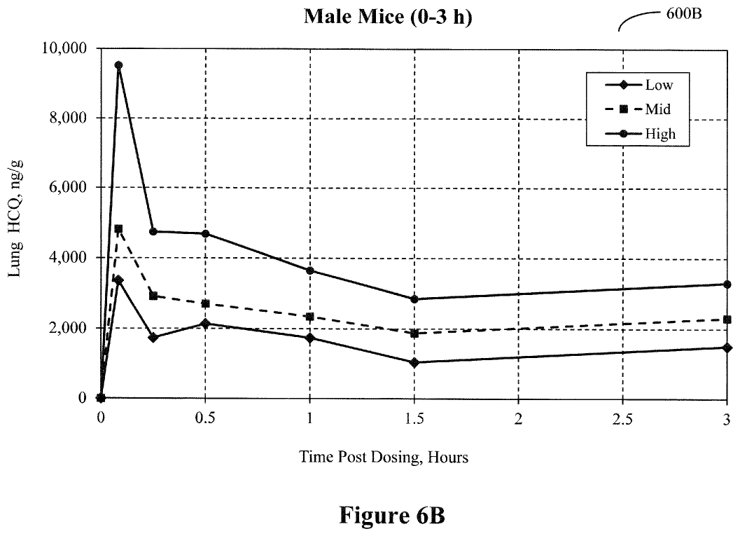

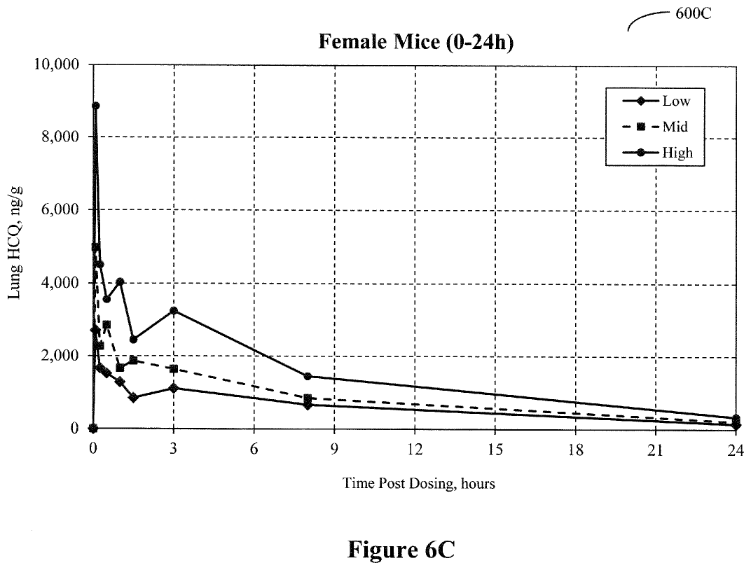

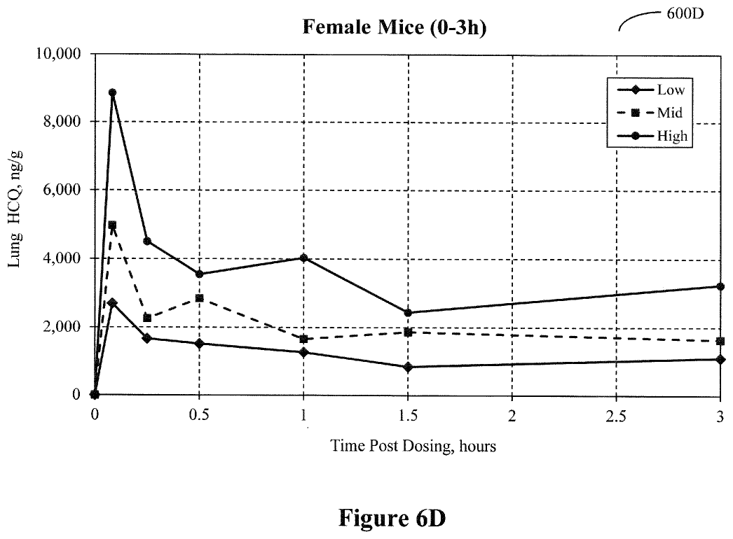

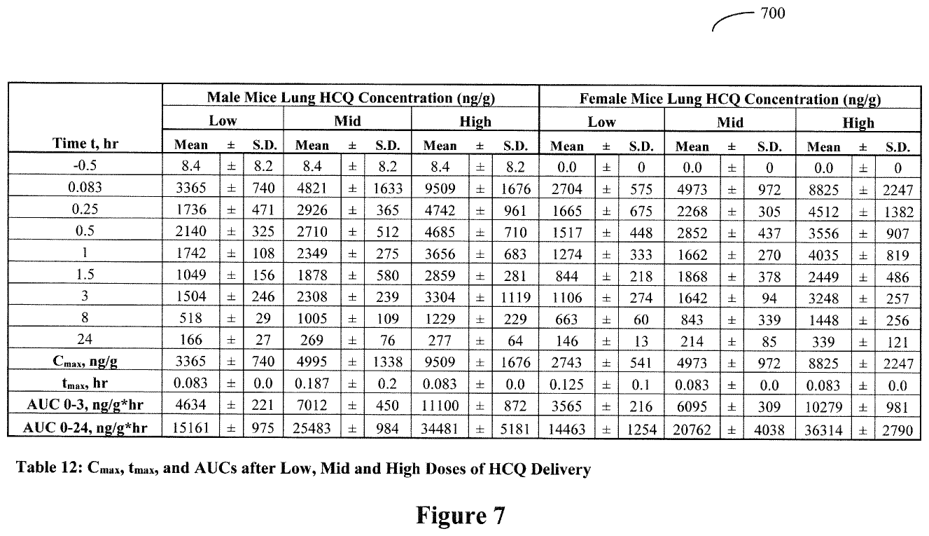

The lung samples of the mice were collected and homogenated at nine time-points of −30′ (30 min before treatment), 5′ 15′, 30′, 1 h, 1.5 h, 3 h, 8 h and 24 h after breathing treatment. For each time point, four mice of each gender were used for each dose level (low, mid, and high). The HCQ concentration delivered into the lungs were analyzed using an LC/MS/MS method. The results are summarized in graphs 600A to 600D of FIGS. 6A to 6D and object 700 of FIG. 7, also referred to as “Table 12”.

As shown in FIGS. 6A to 6D and Table 12 of FIG. 7, the dose response of HCQ was consistent in all study groups. HCQ can quickly reach mouse lungs after inhalation treatment, with the highest levels at the first sampling time, which was 5 minutes post dose. This concentration decreases at 15 minutes, and gradually decreases by about 30-times at 24 hours. The lung HCQ concentrations are well correlated with the doses, with the highest dosing group (16 sprays) having the highest lung deposition levels. The half-life of HCQ in lung was calculated to be 6-8 hours per elimination rate.

### Example 10: Toxicokinetic (TK) Study in Mouse Model

To investigate the toxicokinetic characteristics of the interested HCQ concentration range, a single-dose treatment and a 5-day repeated dose study were both performed in mice using the breathing tank 400. Results are described below.

### Single Dose Study

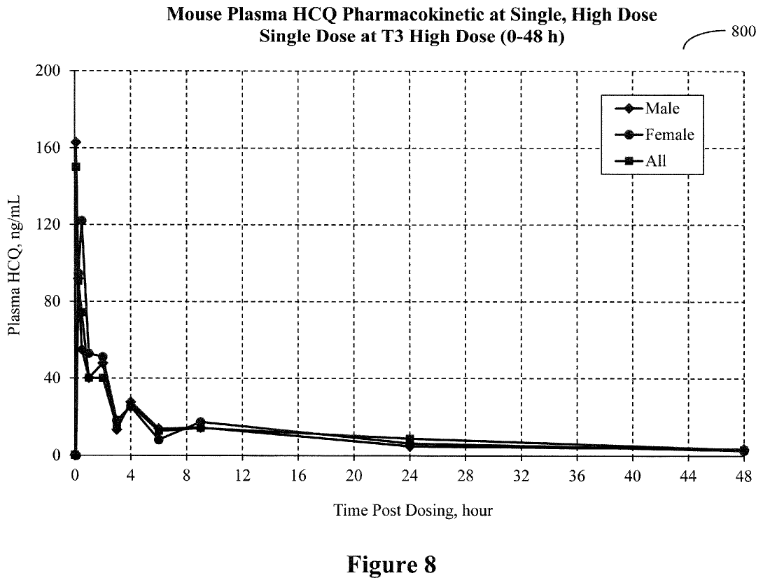

For the single-dose study, 48 mice were treated with the highest dose level (16 sprays) of Formulation 11. Twelve time points were measured: baseline (within one hour before dosing), 5 min, 15 min, 30 min, 1 h, 2 h, 3 h, 4 h, 6 h, 9 h, 24 h and 48 h post dosing. Each animal was sampled twice at two different time points and four mice per gender were used at each time point. As shown by graph 800 in FIG. 8, the average plasma HCQ concentration was at relatively low levels of less than 170 ng/ml.

By comparing with the HCQ concentration data in Example 9 at the same high dose used (16 sprays), the HCQ concentration in mouse plasma is at least 25 folds lower than that observed in mouse lungs (Table 13). The half-life of HCQ in mouse plasma is no less than 4 hours.

TABLE 13
Comparison of HCQ Concentration in Mouse Lung and Plasma

| | | | |
|---|---|---|---|
| | | | Con. Ratio |
| | Lung HCQ Con. | Plasma HCQ | Lung vs |
| Time t, hr. | (ng/g) | Con. (ng/ml) | Plasma |

Male Mice (n = 4)

| | | | |
|---|---|---|---|
| 0.083 | 9509 | 162.9 | 58.4 |
| 0.25 | 4742 | 92.0 | 51.5 |
| 0.5 | 4685 | 55.0 | 85.2 |
| 1 | 3656 | 40.2 | 90.9 |
| 3 | 3304 | 13.2 | 250.3 |
| 24 | 277 | 4.9 | 56.5 |

Female Mice (n = 4)

| | | | |
|---|---|---|---|
| 0.083 | 8825 | 133.2 | 66.3 |
| 0.25 | 4512 | 89.2 | 50.6 |
| 0.5 | 3556 | 93.6 | 38.0 |
| 1 | 4035 | 40.2 | 100.4 |
| 3 | 3248 | 16.8 | 193.3 |
| 24 | 339 | 12.6 | 26.9 |

### 5-Day Repeated Dose

For the 5-day repeated dose toxicokinetic (TK) study, three study arms were used: Arms T1, T2 and T3 which represent low (4 sprays), mid (8 sprays) and high doses (16 sprays) of Formulation 11, respectively. Four male and four female mice were used at each of the 13 TK sampling time points for each repeated-dose arm on both Day-1 and Day-5 treatment. The 13 time points were: baseline (within one hour before dosing), 5 min (0.083 h), 15 min, 30 min, 3 h, 3.083 h, 6 h, 6.083 h, 9 h, 9.083 h, 12 h, 21 h, and 24 h post dosing.

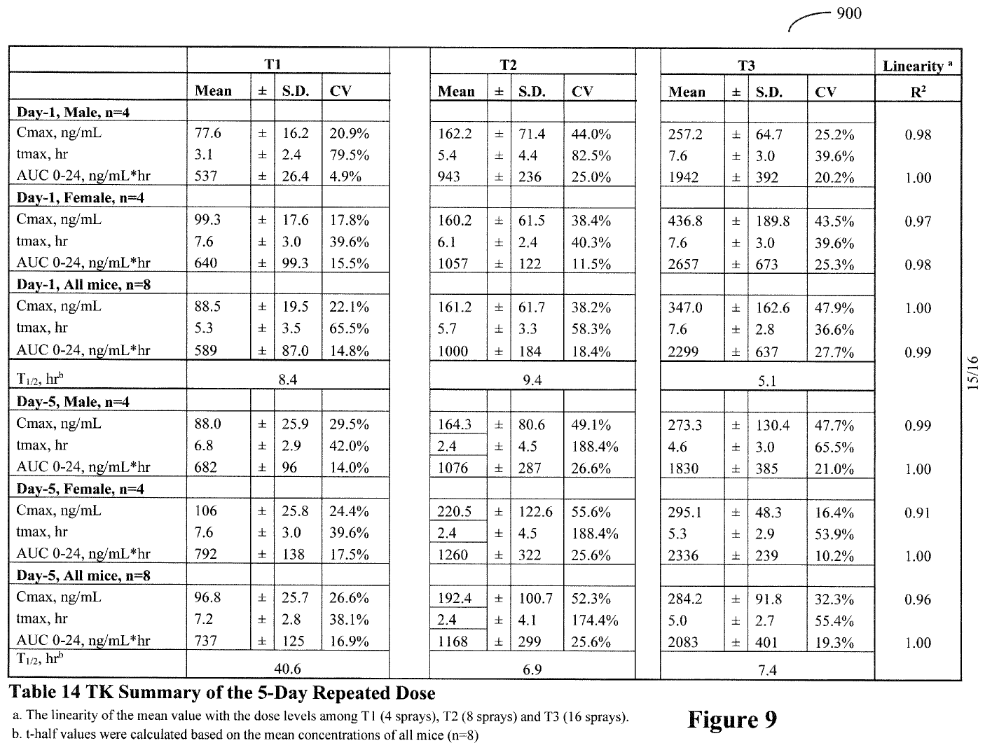

As demonstrated in Table 14 shown as object 900 in FIG. 9, three different systemic exposure levels of medication in mice were reached after undergoing a 5-day repeated-dose nose-only breathing treatment. The dose-exposure is linearly correlated, indicating saturation was not presented in the process of drug absorption, distribution, metabolism, and excretion (ADME). Based on the results shown in Table 14 (FIG. 9), there is no apparent HCQ accumulation in mice plasma after 5 days of repeated-dose treatment.

### Example 11: Determining if HCQ Concentration in Alveolar Lining Fluid (ALF) Exceeds EC₅₀

To evaluate whether the concentration of HCQ delivered into the lungs can exceed the EC₅₀ concentration (0.72-17.31 μM via in vitro¹⁻²) of HCQ for virus inhibition, HCQ in ALF were estimated based on data obtained in Examples 8 and 9, which corresponds to Formulations 5 and 11 respectively described in Table 2. [1. As presented in: Yao X, Ye F, Zhang M, et al. “In Vitro Antiviral Activity and Projection of Optimized Dosing Design of Hydroxychloroquine for the Treatment of Severe Acute Respiratory Syndrome Coronavirus 2 (SARS-CoV-2)”. Clin. Infect Dis. 2020; 71(15):732-739. and 2. Liu J, Cao R, Xu M, et al. Hydroxychloroquine, a less toxic derivative of chloroquine, is effective in inhibiting SARS-CoV-2 infection in vitro. Cell Discov. 2020; 6:16.].

In general, mouse ALF volume can be estimated to be 6 μL based on ALF volume in human (36 ml) and the ratio of lung weight for mouse (average of 0.2 g) and human (1.2 kg) [Refer to Table 11].

### ALF Determination of HCQ in Formulation 5

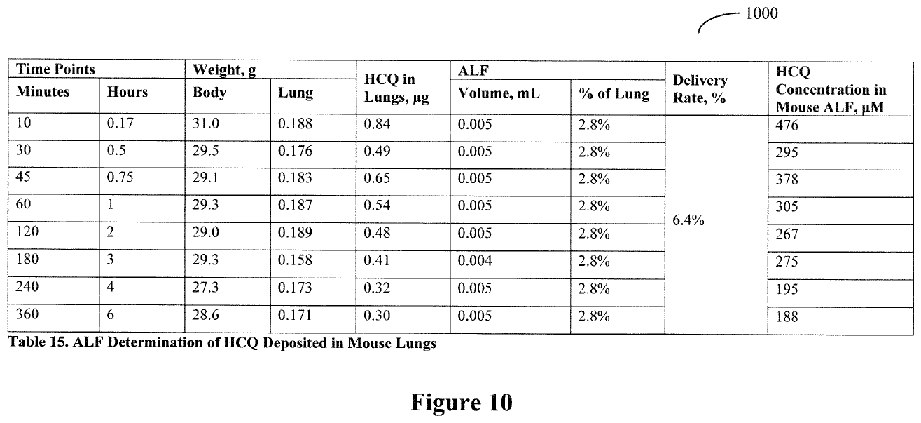

As shown in Table 15 shown as object 1000 in FIG. 10, the ALF HCQ concentration was highest at the first sampling point (10 minutes) which was 476 UM, and remained as high as 188 UM after 6 hours post dosing, which still greatly exceeds the EC₅₀ of HCQ for viral inhibition (0.72-17.31 μM). Overall, a delivery rate of 6.4% of inhaled HCQ reached the mouse lung based on data presented in Table 15 (FIG. 10).

### ALF Determination of HCQ in Formulation 11

ALF HCQ determination was performed using results described in Example 9. For this study, the low dose (4 sprays) was used, which represents the worst-case scenario against the EC₅₀. As shown in Table 16, the ALF HCQ concentration was 322.5 UM at the first sampling time-5 min, and remained as high as 62.8 UM after 8 hours post dosing. This value for low-dose, even after 8 hours, is greatly above the EC₅₀ of HCQ for viral inhibition (0.72-17.31 μM) and the mid-dose (8 sprays) and high dose (16 sprays) are likely even higher.

Overall, a delivery rate of 5.0%-7.1% of inhaled HCQ reached the mouse lung for the three dose levels.

TABLE 16
Mouse ALF HCQ Concentration (Formulation 11, 4 sprays) Calculation

| | | | | | | | | HCQ |
|---|---|---|---|---|---|---|---|---|
| | | | | HCQ in | ALF | | | Concentration |
| Time Points | | Weight, g | | Lungs, | Volume, | % of | Delivery | in Mouse |
| Minutes | Hours | Body | Lung | μg | mL | Lung | Rate, % | ALF, μM |
| 5 | 0.1 | 26.8 | 0.199 | 0.60 | 0.006 | 2.8% | 6.4% | 322.5 |
| 15 | 0.3 | 28.0 | 0.231 | 0.39 | 0.006 | 2.8% | | 180.7 |
| 30 | 0.5 | 27.3 | 0.205 | 0.37 | 0.006 | 2.8% | | 194.4 |
| 60 | 1.0 | 27.9 | 0.206 | 0.31 | 0.006 | 2.8% | | 160.3 |
| 90 | 1.5 | 26.6 | 0.202 | 0.19 | 0.006 | 2.8% | | 100.6 |
| 180 | 3.0 | 27.6 | 0.201 | 0.26 | 0.006 | 2.8% | | 138.7 |
| 480 | 8.0 | 27.8 | 0.197 | 0.12 | 0.006 | 2.8% | | 62.8 |

### Example 12 Local and Systemic Toxicity Study of HCQ Inhalation

This is a 5-day repeated-dose treatment study to evaluate local and systemic toxicity of HCQ in mice after a 14-day recovery period. The study included five treatment arms: low (4 sprays), mid (8 sprays) and high (16 sprays) doses of HCQ Formulation 11, placebo control (Formulation 11 without the HCQ API), and blank control treatment (air). A total of 200 mice were used, in which 40 mice (male:female=1:1) were assigned to each study arm.

Mice were dosed four sessions a day for 5 days, with 3 hours interval between two consecutive treatment sessions. Each session was a 6-minute nose-only inhalation treatment after spraying the designated articles (4, 8 or 16 sprays) into the 21.5 L breathing tank.

After the treatment, 24 mice (male:female=1:1) were sacrificed on Day 6 (or Day-1 post completion of treatment, PCT Day-1) for acute toxicity evaluation and 16 mice (male:female=1:1) were sacrificed on Day 19 (PCT Day-14) for recovery evaluation.

Standard local toxicity evaluation items included: (1) general safety including in-life daily observation and body weight measurement; (2) gross macroscopic observation and organ weight measurement; (3) clinical pathology including hematology and blood chemistry tests; and (4) microscopic histopathological examination. No dose-correlated abnormalities were observed for evaluation items (1)-(3).

Histopathological findings were graded with scores of 1-5 as shown in Table 17 below.

TABLE 17
Severity Grading of Histopathological Findings

| Grade | Score | Description | Definition |
|---|---|---|---|
| Grade 1 | 1 | Minimal | This corresponds to a histopathologic change ranging from |
| | | | inconspicuous to barely noticeable but so minor, small, or |
| | | | infrequent as to warrant no more than the least assignable |
| | | | grade. |
| Grade 2 | 2 | Mild | This corresponds to a histopathologic change that is a |
| | | | noticeable but not a prominent feature of the tissue. |
| Grade 3 | 3 | Moderate | This corresponds to a histopathologic change that is a |
| | | | prominent but not a dominant feature of the tissue |
| Grade 4 | 4 | Marked | This corresponds to a histopathologic change that is a |
| | | | dominant but not an overwhelming feature of the tissue. |
| Grade 5 | 5 | Severe | This corresponds to a histopathologic change that is an |
| | | | overwhelming feature of the tissue. |

For local toxicity, histopathological examination of six organs: nasal cavity (4 turbinate levels), larynx, trachea, branchial lymph nodes, lung and brain were conducted. No dose related abnormalities were observed for all organs at both PCT Day-1 and PCT-Day 14, with the exception of low incidence of minor changes in the nasal turbinates and larynx at PCT-Day 1.

For systemic toxicity evaluation, histopathological evaluation was conducted by a qualified third-party pathology laboratory. A full battery of 55 organs/tissues were examined and evaluated including integumentary, skeletal, muscular, nervous, endocrine, cardiovascular, lymphatic, respiratory, digestive, urinary, and reproductive systems. There were no dose related findings observed for all 55 organs/tissues except for the thymus organ. There were minimal to mild tingible body macrophages seen in the thymus of medication-treated male and female mice at both PCT Day-1 and PCT Day-14, which suggests a possible test article effect. However, these changes in the thymus are not expected to impact functions and health of individual animal.

In summary, based on the overall evaluations on mouse general safety, clinical pathology, gross necropsy and histopathology findings, the no-observed-adverse-effect level (“NOAEL”) dose of HCQ treatment in the mouse model is determined to be the high dose (HCQ: 0.12 mg/kg body weight, 18.1 mg/kg lung weight) or higher for both local toxicity and systemic toxicity evaluation. This dose is 6-fold and 15-fold high to intended human dose for systemic (mg/kg body weight) and local (mg/kg lung weight) dose, respectively. This study demonstrated favorable toxicity, tolerability, and safety profiles of the drug HCQ inhalation.

### Example 13 In Vitro Antiviral Study of HCQ Inhalation

To confirm the antiviral effect of HCQ inhalation, HCQ liquid solution (9.84±0.08 mg/mL) as well as the placebo solution control (0.3 mg/mL Poloxamer 124 in ethanol) was used on Vero-E6-TMPRSS2 cells starting from one hour before virus infection until the end of the assay [Externally contracted Study APS002-1 with Charles River Laboratories (CRL), Portishead, UK].

In the antiviral cell assay, Vero E6-TMPRSS2 cells were seeded into 96 well plates and grown to 80-90% confluency. Compounds were prepared using an 8-point, half-log (˜3-fold) dilution starting at 100 μM. Vehicle (Placebo) and positive control (Remdesivir) wells were included to control for any influence on cell viability. After one hour, compounds were removed and virus was added at a single concentration (100× Median Tissue Culture Infectious Dose (TCID₅₀)), together with equivalent serial dilutions of test compound. Following infection/treatment, cells were incubated for a further hour. After infection, virus/treatment was removed and an overlay medium was added to the cells, together with equivalent serial dilutions of test compound. Cells were visually inspected daily for the appearance of any cytopathic effect (“CPE”). A cell viability (MTT) assay was performed once CPE was complete.

In contrast to that no antiviral activity was shown from placebo control treatment, HCQ (active) showed anti-viral activity with an EC₅₀ value of 11.30 μM (Table 18).

TABLE 18
Antiviral Effect of Tested Compounds in Study APS002-1

| Compound | EC₅₀ (μM) | CC₅₀ (μM) | SI |
|---|---|---|---|
| Vehicle | >100 | >100 | N/A |
| HCQ | 11.30 | >100 | >8.85 |
| Remdesivir | 2.35 | >20 | >8.51 |

SI = selective index (CC₅₀/EC₅₀)

HCQ showed anti-viral activity, with an EC₅₀ value of 11.30 μM. No toxicity was seen, and therefore the selectivity index (SI) was calculated to be >8.85, slightly higher than that of the positive control Remdesivir.

A very high MOI (Multiplicity of Infection) (100×TCID₅₀, equal to about 70 MOI) was used in the antiviral study APS002-1. It has been reported that the EC₅₀ of HCQ was 0.72-4.51 μM when the Vero cells were infected with a low MOI at 0.01^{[1-2]}, which is closer to the natural human infection titer (MOI<<0.01) with SARS-CoV-2 virus. Therefore, in the real world, the EC₅₀ of HCQ in human alveolar lining fluid (ALF) against SARS-CoV-2 virus may be lower than the tested 11.3 μM.

In addition to the Examples described above, aspects of the present disclosure relate to methods for using an MDI, such as the MDI 103 of FIG. 1, to self-administer the disclosed medications (such as anti-viral medications) for treatment of COVID-19 as caused by CoV2 and/or its variants. More particularly, a method is disclosed for delivering a medication for treatment of a pulmonary disease depending on location(s) of infection lesion(s) within a respiratory tract of a patient caused by the pulmonary disease. In some examples, the pulmonary disease is COVID-19 and the virus is SARS-CoV-2. The respiratory tract is defined as comprising an upper airway and a lower airway including alveoli in a relatively deep portion of lungs of the patient. The method includes administering a metered dose of the medication to the patient having the pulmonary disease by a metered dose inhaler (MDI) actuator having a function of controlling drug particle delivery characteristics. The described MDI actuator is MDI actuator 100 of FIG. 1 and the medication is selected from one of the Examples described earlier. The medication is effective for treating the pulmonary disease and a therapeutically effective amount of medication for treating the pulmonary disease is administered by a metered dose of the medication.

The MDI actuator permits self-administration of the medication by the patient. That is, more specifically, the patient is able to actuate the MDI through well-known techniques, such as by compressing a canister toward a metering chamber to release a metered dose of the medication from the canister into the expansion chamber. The metered dose emerges through an actuator as an aerosolized spray which is inhaled by the patient. The MDI actuator 100, more specifically, the orifice 108, has a function of controlling drug particle delivery characteristics for delivery of a metered dose of the medication to the patient (not shown in FIG. 1). More specifically, “a function of controlling drug particle delivery characteristics” is defined by the actuator, which has an orifice 108 with a specific size that aerosolizes a formulated medication with specific particle size distribution and/or delivery characteristics to exit the MDI actuator 100 at a desired speed or velocity. Consequently, by actuation of the MDI actuator 100, disclosed medications can be aerosolized and delivered at a specific amount onto a specific portion of respiratory tract and lungs for a desired use of that medication.

The orifice 108 of the MDI actuator 100 is sized to control the drug particle delivery characteristics of fine and extra fine particles included in a dose. In this way, certain defined quantities of drug particles are delivered within the respiratory tract of the patient based on their respective size to treat infection lesions caused by the pulmonary disease. In some examples, the medication is formulated to include an API, a propellant, a co-solvent, and a surfactant. The API is dissolved in the propellant at a pre-determined ratio, with or without a co-solvent, as described in the above Examples. The medication is administered to the patient via metered-dose inhalation. The method includes dispensing, by the MDI per actuation, the metered dose of the medication at various specific dosage ranges, including from about 0.05 mg to about 1.00 mg, about 0.1 mg to about 0.5 mg, or about 0.1 mg to about 0.4 mg.

In addition, or in the alternative, a method of treating a pulmonary disease caused by a virus in a respiratory tract of a patient depending on location(s) of infection lesion(s) caused by the pulmonary disease is disclosed. In some examples, the pulmonary disease is COVID-19 and the virus is SARS-CoV-2 and/or its variants. The respiratory tract is defined to include an upper airway and a lower airway including alveoli in a relatively deep portion of lungs of the patient. The method includes administering, by a metered dose inhaler (MDI) actuator having a function of controlling drug particle delivery characteristics, a metered dose of an anti-viral medication to the patient. In detail, the described MDI actuator is the MDI actuator 100 of FIG. 1 and the medication is selected from one of the Examples described earlier. More particularly, in some examples, the MDI actuator 100 has an orifice having a diameter of 0.18 mm to 0.25 mm associated with HCQ drug particle delivery.

In this way, the MDI actuator 100, more specifically, the orifice 108 of the MDI actuator, has a function of controlling drug particle delivery characteristics for delivery of at least 70% of a delivered dose of the medication, which includes fine particles of the medication that reach cascade impactor “Stage 3 to Stage 7 and beyond,” including targeting the respiratory tract of the patient, such as from the upper airway to the lower airway including alveoli located in the deep and/or peripheral lungs. “Stage 3 to Stage 7 and beyond” is based on a cascade impactor particle size distribution (PSD) of a respiratory tract. Fine particles of the medication have particle diameter sizes of less than 4.7 μm.

In addition, the MDI actuator 100, more specifically, the orifice 108 of the MDI actuator 100, has a function of controlling drug particle delivery characteristics for delivery of at least 30% of a delivered dose of the medication, which includes extra fine particles of the medication that reach cascade impactor “Stage 6 to Stage 7 and beyond,” including targeting relatively deep regions of lungs of the patient including where alveoli are located in the peripheral lungs. “Stage 6 to Stage 7 and beyond” is based on a cascade impactor particle size distribution (PSD) of a respiratory tract. The extra fine particles of the medication have a PSD of less than 1.1 μm. In some embodiments, a single metered dose of the anti-viral medication from the MDI is capable of delivering aerosolized particles of the anti-viral medication having diameter of less than 1.1 μm.

Delivery of the medication into the respiratory tract of the patient, including a relatively deep portion of lungs of the patient, allows for a reduced systemic dose of the medication, provides a relatively high local concentration of the medication in the respiratory tract, and thereby causes fewer adverse drug events and lowers overdose toxicity risk, while providing increased therapeutic efficacy. The medication is formulated with HCQ, a propellant, a co-solvent, and a surfactant according to the Examples described above. API is dissolved in the propellant at a pre-determined ratio, with or without a co-solvent.

API in the formulation includes HCQ free base or pharmaceutically acceptable salt which can be converted into HCQ free base, which is formed from conversion of HCQ sulfate, and is provided at various concentration levels, such as about 0.25% to about 1.50% (w/w), about 0.40% to about 0.70% (w/w), and about 0.60% to about 0.70% (w/w). The co-solvent includes ethanol, and is provided at various concentration levels, such as at about 1% to about 15% (w/w), about 4.00% to about 8.00% (w/w), or about 5.50% to about 6.50% (w/w). The surfactant includes poly(ethylene glycol)-block-poly(propylene glycol)-block-poly(ethylene glycol) (Poloxamer 124) provided at various concentration levels including at about 0% to 1% (w/w), about 0.01% to about 0.2% (w/w), and about 0.01 to about 0.05% (w/w). The propellant includes 1,1,1,2-Tetrafluoroethane (HFA 134a) provided at various concentration levels including at about 80% to about 97% (w/w), about 93.00% to about 96.00% (w/w), and about 93.00% to about 94.00% (w/w), where “w/w” denotes weight by weight, and is based on a total weight of the medication, which is formulated as a true solution.

In addition, the medication has a favorable half maximal effective concentration (EC₅₀), including that of hydroxychloroquine (HCQ), in a free base thereof, or a pharmaceutically acceptable salt thereof. Half maximal effective concentration (EC₅₀) is the concentration or dose effective in producing 50% of the maximal response. “Favorable half maximal effective concentration (EC₅₀)” is defined as shown in some examples in Table 15, where the estimated alveolar lining fluid (ALF) HCQ concentration was highest at the first sampling point (10 minutes) at 476 μM and remained as high as 188 μM after 6 hours post dosing, all of which still greatly exceeds the EC₅₀ of HCQ for viral inhibition (0.72-17.31 μM). Exceeding the EC₅₀ of HCQ for viral inhibition of CoV2 (0.72-17.31 μM) is understood to be “favorable” by the present disclosure.

The medication was applied to (such as, by being tested in) a non-clinical animal model including mice as shown by the breathing tank 400 of FIG. 4. More specifically, the non-clinical animal model included the MDI actuator inserted into a rodent (mouse) inhaler tank, where multiple mice were placed in mouse restraints with their noses exposed to a main section of the rodent inhaler tank (such as the breathing tank 400) loaded with the medication. In addition, the method includes administering an amount of HCQ according to a human equivalent dose. Delivered particles of HCQ reach the respiratory tract and lungs of treated mice in the non-clinical animal model and demonstrated well-tolerated toxicity and safety profiles. More specifically, “well-tolerated” is demonstrated as having a no-observed-adverse-effect-level (“NOAEL”) dose that is 6-fold and 15-fold higher than the intended human dose. In addition, within a first measurement point taken between 5- and 10-minutes post-dosing via the breathing tank of the non-clinical animal model, at least 5% of the pre-determined amount of HCQ enters into lungs of treated mice in the non-clinical animal model.

The medication delivered into lungs of treated mice in the non-clinical animal model reaches and maintains an estimated alveolar (ALF) HCQ concentration above the HCQ antiviral EC₅₀ concentration against SARS-CoV-2 for no less than 6 hours. In addition, the medication provided to treated mice in the non-clinical animal model has a plasma elimination half-life of not less than 4 hours and has a concentration level in mouse plasma that is at least 25 times lower than that observed in lungs of treated mice in the non-clinical animal model. Concentration levels of the medication in mouse plasma demonstrate no accumulated effect after 5-day repeated doses as indicated by maximum concentration (Cₘₐₓ) and area under the curve (AUC).

While particular embodiments and applications have been illustrated and described, it is to be understood that the invention is not limited to the precise construction and components disclosed herein and that various modifications, changes and variations which will be apparent to those skilled in the art may be made in the arrangement, operation and details of the method and apparatus disclosed herein without departing from the spirit and scope of the present disclosure. Accordingly, the preceding merely illustrates the principles of the invention. Various arrangements may be devised which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method for delivering a medication for treatment of a pulmonary disease depending on location(s) of infection lesion(s) within a respiratory tract of a patient, which includes an upper airway and a lower airway including alveoli in a relatively deep portion of lungs of the patient, the method comprising:
administering, a metered dose of the medication to the patient having the pulmonary disease by a metered dose inhaler (MDI) actuator having a function of controlling drug particle delivery characteristics, wherein:
the medication is effective for treating the pulmonary disease; and
a therapeutically effective amount of medication for treating the pulmonary disease is administered by the metered dose.

2. A method of treating a pulmonary disease caused by viruses in a respiratory tract of a patient, depending on location(s) of infection lesion(s) caused by the pulmonary disease, wherein the respiratory tract includes an upper airway and a lower airway including alveoli in a relatively deep portion of lungs of the patient, of the method comprising:
administering, by a metered dose inhaler (MDI) actuator having a function of controlling drug particle delivery characteristics, an anti-viral medication to the patient, wherein:
the anti-viral medication includes hydroxychloroquine (HCQ) in a free base thereof or a pharmaceutical acceptable salt thereof; and
a therapeutically effective amount of the anti-viral medication for treating the pulmonary disease is administered by a metered dose of the anti-viral medication.

3. The method of claim 2, wherein the pulmonary disease is COVID-19, and viruses include SARS-CoV-2 and/or its variants.

4. The method of claim 2, further comprising:
using the MDI, wherein the MDI actuator has a function of controlling drug particle delivery characteristics for delivery of at least 70% of a delivered dose of the anti-viral medication, which includes fine particles of the anti-viral medication that reach cascade impactor “Stage 3 to Stage 7 and beyond,” targeting the respiratory tract of the patient, from the upper airway to the lower airway and alveoli located in peripheral regions of lungs of the patient, wherein “Stage 3 to Stage 7 and beyond” is based on a cascade impactor particle size distribution of a respiratory tract.

5. The method of claim 4, wherein the fine particles of the anti-viral medication have particle diameter sizes of less than 4.7 μm.

6. The method of claim 2, further comprising:
using the MDI, wherein the MDI actuator has a function of controlling drug particle delivery characteristics for delivery of at least 30% of a delivered dose of the anti-viral medication, which includes extra fine particles of the anti-viral medication that reach cascade impactor “Stage 6 to Stage 7 and beyond” targeting relatively deep regions of lungs of the patient including where alveoli are located in peripheral regions of lungs of the patient, and wherein “Stage 6 to Stage 7 and beyond” is based on a cascade impactor particle size distribution of a respiratory tract.

7. The method of claim 6, wherein the extra fine particles of the anti-viral medication have a particle size diameter of less than 1.1 μm.

8. The method of claim 7, wherein a single metered dose of the anti-viral medication from the MDI is capable of delivering aerosolized particles of the anti-viral medication having particle diameters of less than 1.1 μm.

9. The method of claim 2, wherein the MDI actuator is associated with self-administration of the anti-viral medication to the patient, wherein:
the MDI actuator has a function of controlling drug particle delivery characteristics for delivery of a metered dose of the anti-viral medication to the patient, and
via actuation, the anti-viral medication is aerosolized and delivered with a specific amount, onto a specific portion of respiratory tract and lungs for a desired use of a specific anti-viral medication.

10. The method of claim 2, wherein the MDI actuator further comprises:
an orifice having a diameter of 0.18 mm to 0.25 mm associated with HCQ drug particle delivery.

11. The method of claim 1, wherein the medication further comprises:
an active pharmaceutical ingredient (API) associated with treatment of the pulmonary disease;
a propellant;
a co-solvent, wherein the API is dissolved in the propellant at a pre-determined ratio, with or without a co-solvent; and
a surfactant; wherein:
the medication is administered via metered-dose inhalation.

12. The method of claim 11, wherein:
the API includes HCQ free base or pharmaceutically acceptable salt which can be converted into HCQ base, wherein HCQ free base is from conversion of HCQ sulfate, and is provided at various concentration levels including about 0.25% to about 1.50% (w/w), about 0.40% to about 0.70% (w/w), and about 0.60 to about 0.70% (w/w);
the co-solvent includes ethanol, and is provided at various concentration levels including at about 1% to about 15% (w/w), about 4.00% to about 8.00% (w/w), or about 5.50% to 6.50% (w/w);
the surfactant includes poly(ethylene glycol)-block-poly(propylene glycol)-block-poly(ethylene glycol) (Poloxamer 124) provided at various concentration levels including at about 0% to 1% (w/w), about 0.01% to about 0.2% (w/w), and about 0.01 to about 0.05% (w/w);
the propellant includes 1,1,1,2-Tetrafluoroethane (HFA 134a) provided at various concentration levels including at about 80% to about 97% (w/w), about 93.00% to about 96.00% (w/w), and about 93.00 to about 94.00% (w/w); wherein:
“w/w” denotes weight by weight, and is based on a total weight of the medication, and
the medication is a true solution.

13. The method of claim 2, further comprising:
dispensing, by the MDI per actuation, the metered dose of the anti-viral medication at various dosage ranges, including from about 0.05 mg to about 1.00 mg, about 0.1 mg to about 0.5 mg, or about 0.1 mg to about 0.4 mg.

14. The method of claim 2, wherein the anti-viral medication has a half maximal effective concentration (EC₅₀), including that of hydroxychloroquine (HCQ), in a free base thereof, or a pharmaceutically acceptable salt thereof.

15. The method of claim 2, wherein delivery of the anti-viral medication into relatively deep portion of lungs of the patient allows for a reduced systemic dose of the anti-viral medication, provides a relatively high local concentration of the anti-viral medication in the respiratory tract, and thereby causes fewer adverse drug events and lowers overdose toxicity risk, while providing increased therapeutic efficacy.

16. The method of claim 2, wherein the method was applied to a non-clinical animal model including mice, the method further comprising:
administering an amount of HCQ according to a human equivalent dose, wherein delivered particles of HCQ reach a respiratory tract and lungs of treated mice and demonstrated well-tolerated toxicity and safety profiles.

17. The method of claim 16, wherein within a first measurement point taken between 5 and 10 minutes post-dosing via a breathing tank of the non-clinical animal model, at least 5% of the delivered particles of HCQ enters into lungs of treated mice in the non-clinical animal model.

18. The method of claim 16, wherein the anti-viral medication delivered into lungs of treated mice in the non-clinical animal model reaches and maintains an estimated alveolar lining fluid (ALF) HCQ concentration above the HCQ antiviral EC₅₀ concentration against SARS-CoV-2 for no less than 6 hours.

19. The method of claim 16, wherein the anti-viral medication provided to treated mice in the non-clinical animal model has a plasma elimination half-life of not less than 4 hours.

20. The method of claim 16, wherein the anti-viral medication provided to treated mice in the non-clinical animal model has a concentration level in mouse plasma that is at least 25 times lower than that observed in lungs of treated mice in the non-clinical animal model.

21. The method of claim 16, wherein concentration levels of the anti-viral medication in mouse plasma demonstrate no accumulated effect after 5-day repeated doses as indicated by “maximum concentration” (Cₘₐₓ) and “area under the curve” (AUC).
